(19) United States
(12) Patent Application Publication
(10) Pub. No.: US 2022/0061773 A1
(43) Pub. Date: Mar. 3, 2022

(54) TEST SUPPORT APPARATUS, TEST SUPPORT METHOD, AND TEST SUPPORT PROGRAM

(71) Applicant: FUJIFILM CORPORATION, Tokyo (JP)

(72) Inventors: Kenji NAGAMIYA, Kanagawa (JP); Haruyasu NAKATSUGAWA, Kanagawa (JP); Yasuhisa KANEKO, Kanagawa (JP); Tomohide HIRAGAMI, Kanagawa (JP); Yasuyuki HOSONO, Kanagawa (JP); Nobuya KITAMURA, Kanagawa (JP)

(21) Appl. No.: 17/405,049

(22) Filed: Aug. 18, 2021

(30) Foreign Application Priority Data
Aug. 25, 2020 (JP) 2020-142089
Jan. 12, 2021 (JP) 2021-002928
May 27, 2021 (JP) 2021-089605

(51) Int. Cl. A61B 5/00; G06F 3/0482; G06F 3/0481; G16H 40/63; A61B 5/145

(52) U.S. Cl. CPC A61B 5/7275; G06F 3/0482; G06F 3/04817; G16H 40/63; A61B 5/7221; A61B 5/0004; A61B 5/7435; A61B 5/14532; A61B 5/4866; A61B 5/681; A61B 5/7282

(57) ABSTRACT

A test support apparatus including at least one processor, wherein the processor is configured to determine whether or not there is an abnormal tendency in first biological information of a user by monitoring the first biological information, determine, as plural action candidates to be recommended for the user, plural action candidates including at least one test candidate for acquiring second biological information associated with the first biological information in a case where there is the abnormal tendency in the first biological information, and present the plurality of action candidates to the user.

### CROSS-REFERENCE TO RELATED APPLICATIONS

The present application claims priority under 35 U.S.C. § 119 to Japanese Patent Application No. 2020-142089, filed on Aug. 25, 2020, Japanese Patent Application No. 2021-002928, filed on Jan. 12, 2021, and Japanese Patent Application No. 2021-089605, filed on May 27, 2021, the disclosures of which are incorporated by reference herein.

### BACKGROUND

### Technical Field

The technique of the present disclosure relates to a test support apparatus, a test support method, and a test support program.

### Related Art

In recent years, a method of monitoring biological information such as a pulse, a blood pressure, respiration, an electrocardiogram, maximum oxygen intake, a blood glucose equivalent value, and a body temperature by using a wearable terminal such as a smart watch and managing health improvement and disease prevention based on the monitored biological information is beginning to spread. In addition, by learning artificial intelligence (AI) using big data related to biological information, it is possible to identify diseases and potential diseases with high accuracy.

Further, a method of providing health management information to a user using biological information has been proposed. For example, JP2006-259827A proposes a method of presenting a recommended action plan such as content of an exercise and content of a meal in a case where an abnormality is observed in a monitoring result of a blood glucose value acquired by a wearable terminal. Further, WO2019/188909A proposes a method of determining whether or not a postprandial blood glucose value acquired by a wearable terminal is within a set range, and in a case where the postprandial blood glucose value is not within the set range, notifying a user of a recommended date and time for performing self blood sampling using a blood test kit.

### SUMMARY

However, in the method described in JP2006-259827A, only a recommended action plan is presented to the user. For this reason, in the method described in JP2006-259827A, it is difficult to recognize a current state of biological information with high accuracy in a case where an abnormal tendency is observed in the monitoring result. Further, in the method described in WO2019/188909A, only a recommended date and time for performing self blood sampling using a blood test kit is presented. For this reason, in the method described in WO2019/188909A, even in a case where an abnormal tendency is observed in the biological information as a result of monitoring, an additional test option that a user can take is only self blood sampling using a blood test kit.

The present disclosure has been made in view of the above circumstances, and an object of the present disclosure is to allow a user to take actions including a test according to an abnormal tendency of biological information.

According to an aspect of the present disclosure, there is provided a test support apparatus including: at least one processor configured to determine whether or not there is an abnormal tendency in first biological information of a user by monitoring the first biological information, determine, as plural action candidates to be recommended for the user, plural action candidates including at least one test candidate for acquiring second biological information associated with the first biological information in a case where there is the abnormal tendency in the first biological information, and present the plurality of action candidates to the user.

“Presenting” means allowing the user to recognize the action candidates, and includes displaying the action candidates on a display, outputting the action candidates from the apparatus so as to display the action candidates on a display, or outputting the action candidates by voice.

In the test support apparatus according to the aspect of the present disclosure, the action candidates may include the test candidate in which the second biological information is acquired with accuracy higher than accuracy of the first biological information.

In the test support apparatus according to the aspect of the present disclosure, the processor may be configured to receive designation of at least one item serving as an index in a case where the user selects the presented action candidates, and present the action candidates in a display form according to the designated item.

In the test support apparatus according to the aspect of the present disclosure, the item may serve as an index in a case where the user selects the test candidate included in the presented action candidates, and the processor may be configured to present the test candidate in a display form according to the designated item.

In the test support apparatus according to the aspect of the present disclosure, the processor may be configured to present, to the user, a question for determining a display position of the test candidate on at least one scale according to the designated at least one item, determine the display position of the test candidate on the scale according to an answer to the question, and present, to the user, the test candidate in a display form in which an icon representing the test candidate is displayed at the determined display position on the scale.

In the test support apparatus according to the aspect of the present disclosure, the processor may be configured to receive additional designation of the item by the user after the icon representing the test candidate is displayed, and update the display form of the icon representing the test candidate in a case where designation of the item is added.

In the test support apparatus according to the aspect of the present disclosure, the processor may be configured to repeatedly perform receiving of additional designation of the item, presenting of a question according to the added item, and updating of the display form of the test candidate until the test candidate is selected by the user.

In the test support apparatus according to the aspect of the present disclosure, the item may include at least one of a cost required for a test, a sense of stability in a case where a sample is collected, a degree of restraint in a test, invasiveness or non-invasiveness, a time required to obtain a test result, whether to include a risk check for various diseases, or test accuracy.

In the test support apparatus according to the aspect of the present disclosure, the processor may be configured to selectively determine the test candidate from plural test methods including designation of a type of a sample and used for acquiring the second biological information from the sample.

In the test support apparatus according to the aspect of the present disclosure, the processor may be configured to determine the test candidate according to a degree of the abnormal tendency.

In the test support apparatus according to the aspect of the present disclosure, the processor may be configured to determine the test candidate according to an elapsed time from a time when it is determined that there is the abnormal tendency in the first biological information to a current time.

In the test support apparatus according to the aspect of the present disclosure, the processor may be configured to determine the test candidate according to a risk factor that the user has.

In the test support apparatus according to the aspect of the present disclosure, the processor may be configured to present, to the user, statistical information of the test candidate which is previously selected.

In the test support apparatus according to the aspect of the present disclosure, the first biological information may be a blood glucose equivalent value that correlates with a blood glucose value, and the second biological information may include at least one of a blood glucose value or HbA1c.

In the test support apparatus according to the aspect of the present disclosure, the abnormal tendency may be determined based on a postprandial hyperglycemic spike.

In the test support apparatus according to the aspect of the present disclosure, the processor may be configured to determine the test candidate based on meal content of the user.

In the test support apparatus according to the aspect of the present disclosure, the first biological information may be at least one of a heart rate, a blood pressure, respiration, an electrocardiogram, maximum oxygen intake, arterial oxygen saturation, or a body temperature, and the second biological information may be a diagnosis result of a disease of the user.

In the test support apparatus according to the aspect of the present disclosure, the processor may be configured to perform a notification according to a monitoring result of the first biological information in a case where it is determined that there is no abnormal tendency in the first biological information.

In the test support apparatus according to the aspect of the present disclosure, the first biological information may be acquired by a measurement device that the user wears.

In the test support apparatus according to the aspect of the present disclosure, the measurement device may be a wearable measurement device.

In the test support apparatus according to the aspect of the present disclosure, the processor may be configured to perform a notification for urging the user to wear the measurement device for a longer time in a case where whether or not there is an abnormal tendency in the first biological information is not determined.

The test support apparatus according to the aspect of the present disclosure may further include a sensor that acquires the first biological information.

In the test support apparatus according to the aspect of the present disclosure, the test support apparatus may be a wearable apparatus.

In the test support apparatus according to the aspect of the present disclosure, the action candidates may further include a recommended action for the user.

In the test support apparatus according to the aspect of the present disclosure, the processor may be configured to acquire the second biological information, and determine whether to continue monitoring of the first biological information according to the acquired second biological information.

According to another aspect of the present disclosure, there is provided a test support method including: determining whether or not there is an abnormal tendency in first biological information of a user by monitoring the first biological information; determining, as plural action candidates to be recommended for the user, plural action candidates including at least one test candidate for acquiring second biological information associated with the first biological information in a case where there is the abnormal tendency in the first biological information; and presenting the plurality of action candidates to the user.

According to still another aspect of the present disclosure, there is provided a test support program causing a computer to execute: a procedure of determining whether or not there is an abnormal tendency in first biological information of a user by monitoring the first biological information; a procedure of determining, as plural action candidates to be recommended for the user, plural action candidates including at least one test candidate for acquiring second biological information associated with the first biological information in a case where there is the abnormal tendency in the first biological information; and a procedure of presenting the plurality of action candidates to the user.

According to the present disclosure, the user can take actions including a test according to an abnormal tendency of biological information.

### BRIEF DESCRIPTION OF THE DRAWINGS

FIG. 1 is a schematic diagram illustrating a configuration of a test support system to which a test support apparatus according to an embodiment of the present disclosure is applied.

FIG. 2 is a hardware configuration diagram of the test support apparatus according to the present embodiment.

FIG. 3 is a hardware configuration diagram of a wristwatch-type measurement device.

FIG. 4 is a hardware configuration diagram of the measurement device for measuring glucose in an interstitial fluid.

FIG. 5 is a functional configuration diagram of the test support apparatus according to the present embodiment.

FIG. 6 is a graph illustrating daily variations in blood glucose of a patient with type 2 diabetes.

FIG. 7 is a table illustrating test methods for biological information associated with a blood glucose equivalent value.

FIG. 8 is a flowchart illustrating processing performed in the present embodiment.

FIG. 9 is a flowchart illustrating processing performed in the present embodiment.

FIG. 10 is a diagram illustrating a notification screen in a case where there is no abnormal tendency in the blood glucose equivalent value.

FIG. 11 is a diagram illustrating an action candidate presentation screen.

FIG. 12 is a diagram illustrating an action candidate presentation screen on which a description of a selected test candidate is displayed.

FIG. 13 is a diagram illustrating an action candidate presentation screen on which statistical information of a test candidate which is previously selected is displayed.

FIG. 14 is a diagram illustrating an action candidate presentation screen on which contents of recommended actions are displayed.

FIG. 15 is a diagram illustrating an item list screen including an item serving as an index in a case where a user selects a test candidate.

FIG. 16 is a diagram illustrating a question presentation screen.

FIG. 17 is a diagram illustrating a presentation screen for presenting test candidates to a user in a display form in which test candidate icons are displayed on a scale.

FIG. 18 is a diagram illustrating an item list screen including an item serving as an index in a case where a user selects a test candidate.

FIG. 19 is a diagram illustrating a question presentation screen.

FIG. 20 is a diagram illustrating a presentation screen for presenting test candidates to a user in a display form in which test candidate icons are displayed on a scale in a case where two items are selected.

FIG. 21 is a diagram illustrating a guide screen for guiding a website that sells a blood glucose measurement device.

FIG. 22 is a diagram illustrating a guide screen for guiding, to a user, a hospital at which a glucose tolerance test can be performed.

FIG. 23 is a diagram illustrating a guide screen for a next test.

FIG. 24 is a diagram illustrating a screen for inquiring of a user about a result of a urine glucose test.

FIG. 25 is a diagram illustrating a notification screen for urging a user to wear a measurement device for a longer time.

FIG. 26 is a graph illustrating a comparison result between a monitoring result of a blood glucose equivalent value and a test result obtained by blood sampling of a user.

FIG. 27 is a schematic graph illustrating changes in heart rate variability and the like of a patient who is infected with new coronavirus infection.

FIG. 28 is a table illustrating test methods related to new coronavirus infection.

FIG. 29 is a diagram illustrating a presentation screen for presenting test candidates to a user in a display form in which test candidate icons are displayed on a scale in a case where two items are selected.

FIG. 30 is a diagram illustrating a presentation screen for presenting test candidates to a user in a display form in which test candidate icons are displayed on a scale in a case where two items are selected.

FIG. 31 is a diagram illustrating a question presentation screen.

### DESCRIPTION OF EMBODIMENTS

### First Embodiment

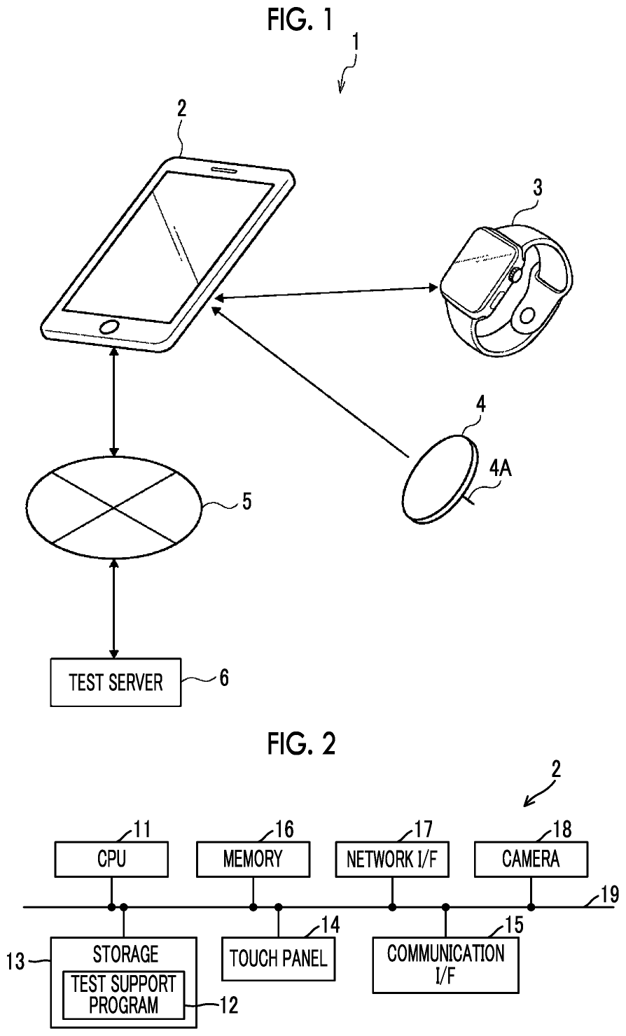

Hereinafter, a first embodiment of the present disclosure will be described with reference to the drawings. FIG. 1 is a schematic diagram illustrating a configuration of a test support system to which a test support apparatus according to a first embodiment of the present disclosure is applied. As illustrated in FIG. 1, the test support system 1 according to the present embodiment includes a mobile terminal 2 such as a smartphone and measurement devices 3 and 4. The mobile terminal 2 and the measurement devices 3 and 4 can communicate with each other by short-range wireless communication such as Bluetooth (registered trademark). The mobile terminal 2 is connected to a test server 6 via a wired/wireless network 5 such that communication can be performed. The mobile terminal 2 is an example of the test support apparatus according to the present embodiment.

The measurement device 3 is a wristwatch-type wearable terminal such as a smart watch, and has a function of constantly measuring a blood glucose equivalent value that correlates with a blood glucose value of a user. The blood glucose equivalent value is biological information that correlates with the blood glucose value and is measured by a method that does not rely on blood sampling. The constant measurement means that the blood glucose equivalent value is automatically measured at a predetermined time interval, for example, 15 minutes, 30 minutes, or the like, without a measurement instruction from a user. The measurement device 3 may measure the blood glucose equivalent value even in a case where an instruction from a user is input while constantly measuring the blood glucose equivalent value. Further, the measurement device 3 may be a device that is worn by the user only at the time of measurement, such as a finger clip.

Further, the measurement device 3 is a non-invasive measurement device for the blood glucose equivalent value, and obtains the blood glucose equivalent value by, for example, irradiating the user with infrared rays and analyzing a signal emitted by glucose in blood. Alternatively, the measurement device 3 measures an electrocardiogram of the user, and obtains a blood glucose equivalent value that correlates with changes in the electrocardiogram. The measurement device 3 transmits the obtained blood glucose equivalent value to the mobile terminal 2.

The measurement device 4 is an invasive measurement device for the blood glucose equivalent value. For example, the measurement device 4 is attached to the user, constantly measures a glucose concentration in an interstitial fluid under epidermis of the user, and transmits the measured glucose concentration to the mobile terminal 2. For this reason, the measurement device 4 includes a needle-shaped filament 4A that is inserted under the epidermis of the user. The glucose concentration correlates with the blood glucose value, and thus the glucose concentration corresponds to the blood glucose equivalent value.

The blood glucose equivalent value measured by the measurement devices 3 and 4 is an example of first biological information. The user may possess any one of the measurement device 3 or the measurement device 4.

The mobile terminal 2 determines whether or not there is an abnormal tendency in the blood glucose equivalent value by monitoring the blood glucose equivalent value measured by the measurement devices 3 and 4. In a case where there is an abnormal tendency in the blood glucose equivalent value, the mobile terminal 2 presents plural action candidates to be recommended to the user. The action candidates include at least one test candidate for acquiring biological information associated with the blood glucose equivalent value measured by the measurement devices 3 and 4.

Here, in the present embodiment, the mobile terminal 2 determines whether or not there is an abnormal tendency in the blood glucose equivalent value by determining whether or not the blood glucose equivalent value is in a state of a postprandial hyperglycemic spike. The postprandial hyperglycemic spike is a symptom observed in an early stage of diabetes, and is a symptom in which the blood glucose value significantly increases approximately one hour to two hours after a meal even in a case where a fasting blood glucose value is within a normal range. In a case where the postprandial hyperglycemic spike is neglected, blood vessels are damaged and complications of arteriosclerosis and diabetes are more likely to progress. It is also considered that complications such as myocardial infarction, angina, and stroke are more likely to progress. In the present embodiment, the blood glucose equivalent value is monitored, and in a case where it is determined that there is an abnormal tendency in the blood glucose equivalent value, the plurality of action candidates including the test candidate to be recommended for the user are presented to the user. Thereby, it is possible to allow the user to take a test in which biological information for accurately recognizing the postprandial hyperglycemic spike can be acquired.

The network 5 is a wide area network (WAN) that widely connects the mobile terminal 2 and the test server 6 via a public line network or a dedicated line network.

The test server 6 is provided in a test center that supports a test related to the blood glucose value. The test server 6 has a function of providing a test support program according to the present embodiment to the mobile terminal 2 or a function of providing information which is required when the mobile terminal 2 executes the test support program to the mobile terminal 2. In the test server 6, a software program for providing server functions to a general-purpose computer is installed.

The test center provides, to the user, various support for the test related to the blood glucose value. For example, the test center provides a test support program according to the present embodiment. Thus, the test center supports the user to select a test method. In addition, in a case where the user selects a test method, the test center also supports purchase of devices and test kits required for the selected test, and supports hospital test reservations. Further, the test center also performs a test using a specimen, which is obtained by self blood sampling of a user and is delivered by the user.

Next, the test support apparatus according to the present embodiment will be described. A hardware configuration of the test support apparatus according to the present embodiment will be described with reference to FIG. 2. As illustrated in FIG. 2, the mobile terminal 2 as the test support apparatus according to the present embodiment is a portable computer such as a smartphone, and includes a central processing unit (CPU) 11, a non-volatile storage 13, and a memory 16 as a transitory storage area. Further, the mobile terminal 2 includes a touch panel 14, a communication interface (I/F) 15 for short-range wireless communication, and a network I/F 17 wirelessly connected to the network 5. Further, the mobile terminal 2 includes a camera 18. The CPU 11, the storage 13, the touch panel 14, the communication I/F 15, the memory 16, the network I/F 17, and the camera 18 are connected to a bus 19. The CPU 11 is an example of a processor according to the present disclosure.

The storage 13 is realized by a solid state drive (SSD), a flash memory, or the like. A test support program 12 installed in the mobile terminal 2 is stored in the storage 13 as a storage medium. The CPU 11 reads out the test support program 12 from the storage 13, develops the test support program 12 in the memory 16, and executes the developed test support program 12.

The touch panel 14 is configured with a liquid crystal display, an organic EL, or the like, and performs various displays related to processing performed by the mobile terminal 2. The touch panel 14 also has a function as an input device for inputting various information to the mobile terminal 2.

The camera 18 acquires an image of a meal of the user by capturing the meal of the user according to, for example, an instruction of the user. The acquired image of the meal is stored in the storage 13. Alternatively, as described later, the acquired image of the meal is transmitted to the test server 6 together with the monitoring result of the blood glucose equivalent value according to an instruction of the user or without waiting for an instruction of the user.

The test support program 12 is stored in the test server 6 in a state of being accessible from the outside, and is downloaded and installed in the mobile terminal 2 in response to a request.

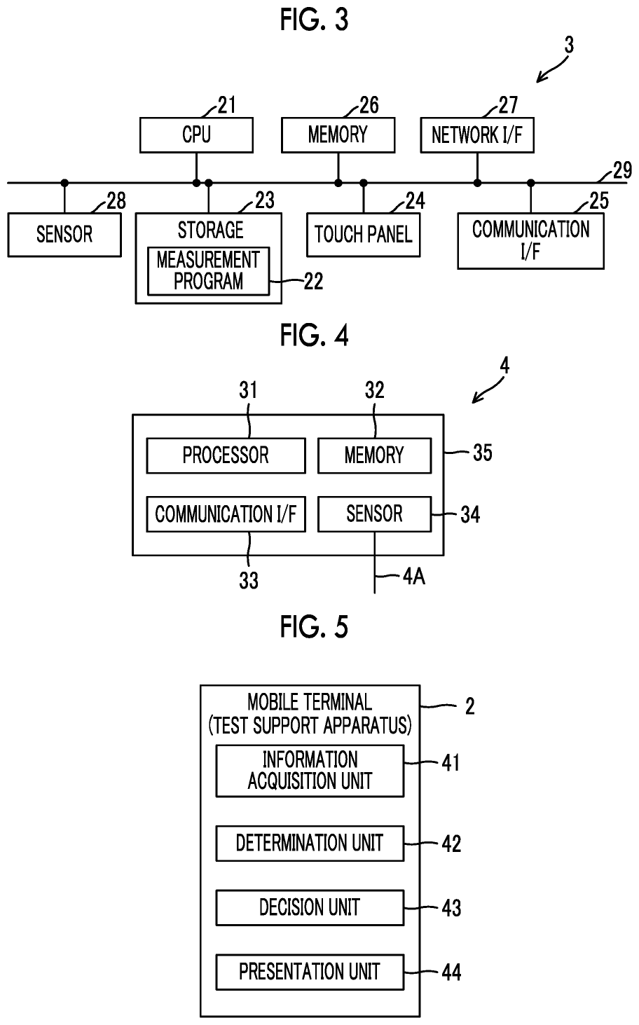

Next, the measurement device will be described. FIG. 3 illustrates a hardware configuration of the measurement device 3. The measurement device 3 is a wristwatch-type computer, and as illustrated in FIG. 3, includes a CPU 21, a non-volatile storage 23, and a memory 26 as a transitory storage area. Further, the measurement device 3 includes a touch panel 24, a communication I/F 25 for short-range wireless communication, a network I/F 27 wirelessly connected to an external network (not illustrated), and a sensor 28. The CPU 21, the storage 23, the touch panel 24, the communication I/F 25, the memory 26, the network I/F 27, and the sensor 28 are connected to a bus 29.

The storage 23 is realized by an SSD, a flash memory, or the like. A measurement program 22, which is installed in the measurement device 3 and is used to measure the blood glucose equivalent value, is stored in the storage 23 as a storage medium. The CPU 21 reads out the measurement program 22 from the storage 23, develops the measurement program 22 in the memory 26, and executes the developed measurement program 22.

The touch panel 24 is configured with a liquid crystal display, an organic EL, or the like, and performs various displays related to processing performed by the measurement device 3. The touch panel 24 also has a function as an input device for inputting various information to the measurement device 3.

The sensor 28 includes, for example, an infrared light source and an infrared detector, and detects a signal emitted by glucose in blood by irradiating a user who wears the measurement device 3 with infrared rays. The signal detected by the sensor 28 is analyzed by the CPU 21 that executes the measurement program 22, and thus the blood glucose equivalent value is obtained. Further, the sensor 28 may measure an electrocardiogram. In this case, the electrocardiogram is analyzed by the measurement program 22, and thus the blood glucose equivalent value is obtained.

The measurement program 22 is stored in the test server 6 in a state of being accessible from the outside, and is downloaded and installed in the measurement device 3 in response to a request. Alternatively, the measurement program 22 is downloaded in the mobile terminal 2, and then is downloaded and installed in the measurement device 3 via short-range wireless communication with the mobile terminal 2.

In the measurement device 3, the sensor 28 detects a signal emitted by glucose in blood at a predetermined time interval. The CPU 21 obtains the blood glucose equivalent value by analyzing the signal by the measurement program 22. Further, the CPU 21 transmits the obtained blood glucose equivalent value from the communication I/F 25 to the mobile terminal 2.

Next, the measurement device 4 will be described. FIG. 4 is a hardware configuration diagram of the measurement device 4. As illustrated in FIG. 4, the measurement device 4 is, for example, a measurement device described in JP2016-520379A, which is attached to a human body and measures, as a blood glucose equivalent value, a glucose concentration in an interstitial fluid under epidermis. The measurement device 4 includes a processor 31, a memory 32 as a transitory storage area, a communication I/F 33 for short-range wireless communication, and a sensor 34. A needle-shaped filament 4A that is inserted under the epidermis is connected to the sensor 34. The processor 31, the memory 32, the communication I/F 33, and the sensor 34 are configured with an application specific integrated circuit (ASIC) 35 for measuring glucose in the interstitial fluid.

In the measurement device 4, the sensor 34 detects a signal indicating a glucose concentration in the interstitial fluid under the epidermis at a predetermined time interval or according to a measurement instruction by the user. The processor 31 obtains a glucose concentration by analyzing the signal. Further, the processor 31 transmits the obtained glucose concentration from the communication I/F 33 to the mobile terminal 2.

Next, a functional configuration of the test support apparatus according to the present embodiment will be described. FIG. 5 is a diagram illustrating a functional configuration of the test support apparatus according to the present embodiment, that is, the mobile terminal 2. As illustrated in FIG. 5, the mobile terminal 2 includes an information acquisition unit 41, a determination unit 42, a decision unit 43, and a presentation unit 44. The CPU 11 functions as the information acquisition unit 41, the determination unit 42, the decision unit 43, and the presentation unit 44 by executing the test support program 12. In the following description, it is assumed that the user possesses the measurement device 3 and monitors the blood glucose equivalent value measured by the measurement device 3 by using the mobile terminal 2.

The information acquisition unit 41 acquires the blood glucose equivalent value transmitted from the communication I/F 25 of the measurement device 3 by receiving the blood glucose equivalent value by the communication I/F 15. Since the blood glucose equivalent value is basically transmitted from the measurement device 3 at all times, that is, at predetermined time intervals, the information acquisition unit 41 constantly acquires the blood glucose equivalent value. The blood glucose equivalent value acquired by the information acquisition unit 41 is stored in the storage 13 in association with the acquisition date and time.

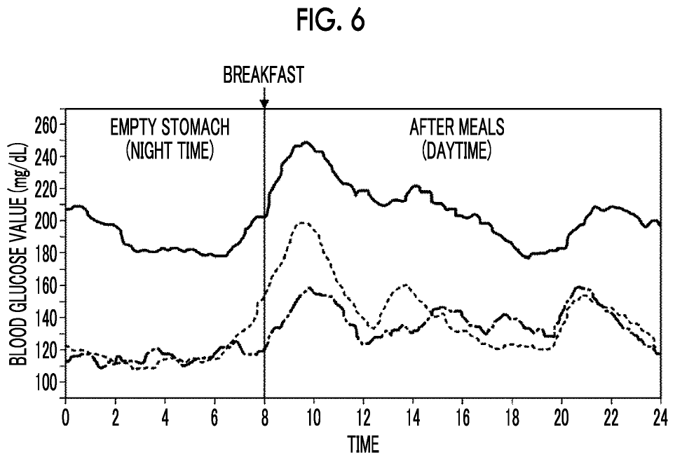

The determination unit 42 determines whether or not there is an abnormal tendency in the blood glucose equivalent value by monitoring the blood glucose equivalent value acquired by the information acquisition unit 41. Specifically, the determination unit 42 determines whether or not the blood glucose equivalent value is in a state of a postprandial hyperglycemic spike. Here, postprandial hyperglycemia will be described. FIG. 6 is a graph illustrating daily variations in blood glucose of a patient with type 2 diabetes. In FIG. 6, a horizontal axis represents a time of one day (24 hours), and a vertical axis represents a blood glucose value (mg/dL). A solid line represents a blood glucose value of a patient whose hemoglobin A1c (HbA1c) is equal to or higher than 9%. A broken line represents a blood glucose value of a patient whose HbA1c is equal to or higher than 7% and lower than 8%. A one-dot line represents a blood glucose value of a patient whose HbA1c is equal to or higher than 6.5% and lower than 7%. Further, FIG. 6 illustrates a variation in the blood glucose value in a case where the patient eats breakfast at around 8:00, eats lunch at around 12:00, and eats dinner at around 19:00. HbA1c is glycated hemoglobin in which hemoglobin as an erythrocyte component in blood is bonded with glucose. HbA1c represents a variation in the blood glucose for 1 to 2 months.

As illustrated in FIG. 6, the blood glucose value increases after meal regardless of a value of HbA1c. On the other hand, as HbA1c is higher, the postprandial hyperglycemic spike indicating that the blood glucose value greatly increases is particularly remarkable after breakfast. In order to make the postprandial hyperglycemic spike after breakfast easy to understand, in FIG. 6, a line is marked at a position indicating 8:00.

The determination unit 42 monitors the blood glucose equivalent value transmitted from the measurement device 3, and in a case where a temporal variation in the blood glucose equivalent value is equal to or larger than a predetermined threshold value Th1, determines that there is an abnormal tendency in the blood glucose equivalent value. In the present embodiment, the temporal variation is a difference between the blood glucose equivalent value measured this time and the latest blood glucose equivalent value. Further, the temporal variation in the blood glucose equivalent value may be obtained by considering the blood glucose equivalent values acquired at plural latest timings.

In the present embodiment, in a case where the variation in the blood glucose equivalent value is equal to or larger than a threshold value Th2, the determination unit 42 determines that the postprandial hyperglycemic spike is large. Here, Th2>Th1. In a case where the variation in the blood glucose equivalent value is equal to or larger than a threshold value Th3 and smaller than the threshold value Th2, the determination unit 42 determines that the postprandial hyperglycemic spike is small. Here, Th2>Th3>Th1. In a case where the variation in the blood glucose equivalent value is equal to or larger than the threshold value Th1 and smaller than the threshold value Th3, the determination unit 42 determines that caution is required because the blood glucose value is high. In a case where the fasting blood glucose value is equal to or larger than a predetermined threshold value Th4, the determination unit 42 determines that the fasting blood glucose is high.

In a case where it is determined that there is no abnormal tendency in the blood glucose equivalent value, the determination unit 42 notifies the user of a fact that there is no abnormal tendency in the blood glucose equivalent value. The notification will be described later.

On the other hand, in order to assist the determination in the determination unit 42, a meal time may be transmitted from the measurement device 3 to the mobile terminal 2 by an operation of the user. Alternatively, the user may input a meal time to the mobile terminal 2. Alternatively, an image of the meal may be captured by the camera 18 of the mobile terminal 2. In this case, the capturing time may be set as a meal time and may be used to assist the determination. Thereby, it is possible to determine whether or not there is an increase tendency in the blood glucose equivalent value based on an accurate meal time.

In a case where the determination unit 42 determines that there is an abnormal tendency in the blood glucose equivalent value, the decision unit 43 decides plural action candidates to be recommended for the user. The action candidates include at least one test candidate for acquiring biological information associated with the blood glucose equivalent value. In addition, the decision unit 43 decides a test candidate according to a degree of the abnormal tendency.

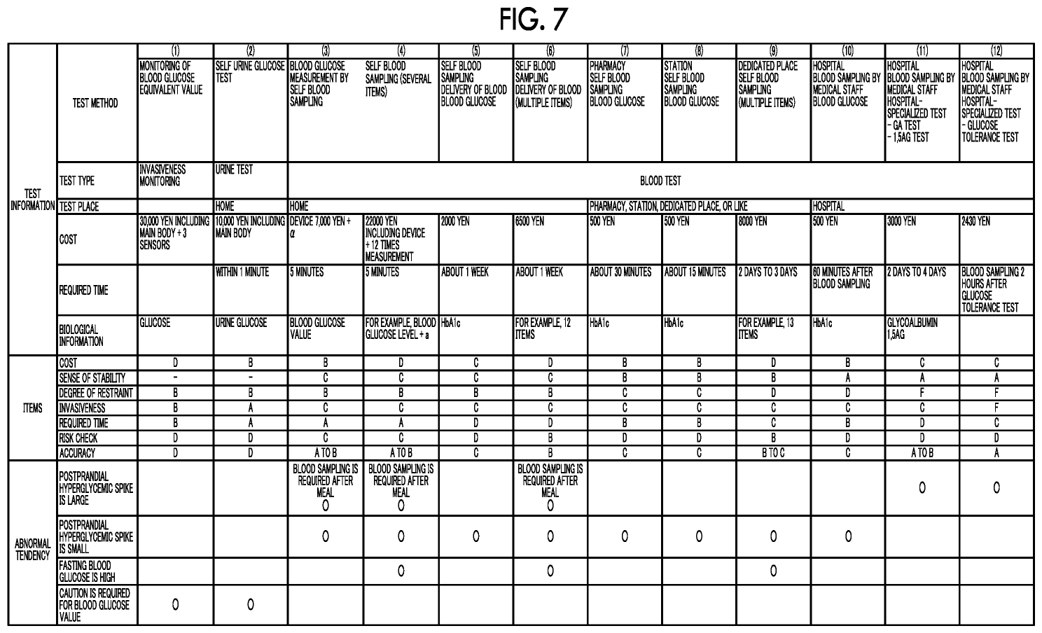

FIG. 7 is a table illustrating test methods for biological information associated with the blood glucose equivalent value. In FIG. 7, for 12-type test methods (1) to (12), a test type, a test place, a cost, a required time, and biological information to be measured are illustrated as test information.

In the test method (1), monitoring of the blood glucose equivalent value is a test for monitoring the blood glucose equivalent value using the measurement device 4. In the test method (2), a self urine glucose test is a test for detecting urine glucose contained in the user's urine using a dedicated measurement device. The urine glucose is glucose that is excreted in the urine. There is also a urine glucose test using a test paper. In the test methods (3) to (6), self blood sampling test methods performed at home include test methods (test methods (3) and (4)) in which tests are completed at home and test methods (test methods (5) and (6)) in which blood obtained by self blood sampling is delivered to a test center as an external test agency. The test method (3) is a test for measuring the blood glucose value using a blood glucose measurement device (self monitoring of blood glucose (SMBG)). An option (several items) described in the test method (4) means that the test method is a test method in which some tests are performed in addition to the blood glucose value. In FIG. 7, some test methods are indicated by “+a”.

The test methods (1) and (2) have an accuracy lower than an accuracy of the blood sampling test. On the other hand, by referring to test results by the test methods (1) and (2) in addition to the blood glucose equivalent value by the measurement device 3, as compared with a case where only the blood glucose equivalent value by the measurement device 3 is used, a tendency of the user's blood glucose can be recognized more accurately.

In the test methods (7) to (9), tests in a pharmacy and the like are blood tests using a test device provided at a pharmacy, a station, or a dedicated place (for example, a convenience store). In the test methods (10) to (12), tests performed in a hospital are tests in which the user goes to a hospital and a medical staff performs blood sampling. Among the tests performed in a hospital, in the test method (11), a GA test is a glycoalbumin test, and a 1,5AG test is a 1,5-anhydro-D-glucitol test. The glucoalbumin is a combination of albumin, which is a kind of protein in serum, and glucose, and is biological information by which a state of the blood glucose value before one week to two weeks can be recognized. 1,5AG is sugar in blood, and is the second highest amount of sugar after glucose. By measuring 1,5AG as biological information, a state of the blood glucose value for previous several days can be recognized. The GA test, the 1,5AG test, and a glucose tolerance test are all tests specialized for postprandial hyperglycemia. For this reason, “specialized test” is indicated in a test method column. Further, in the test methods (6) and (9), 12 items and 13 items are described in a biological information column, and this means that the numbers of the test items are 12 and 13. It is assumed that the test items include a test for measuring a blood glucose value and HbA1c as biological information.

The glucose, the urine glucose, the blood glucose value, HbA1c, glycoalbumin, and 1,5AG, which are biological information obtained by the test methods (1) to (12), are examples of second biological information associated with the blood glucose equivalent value.

FIG. 7 illustrates various items serving as indexes in a case where the user selects the presented test candidate. Specifically, various items include an item for a cost required for a test (cost), an item for a sense of stability in a case where a sample is collected (sense of stability), an item for a degree of restraint in a test (degree of restraint), an item for invasiveness or non-invasiveness (invasiveness), an item for a time required to obtain a test result (required time), an item for whether to include a risk check for various diseases (risk check), and an item for a test accuracy (accuracy). In a column of each item, a degree of preference is indicated for each test method. The degree of preference is represented by symbols A, B, C, D, and F in order of preference. A is very preferable, B is preferable, C is neither, D is not preferable, and F is very unpreferable.

In the cost, as the cost is cheaper, the degree of preference is larger. In the sense of stability, as a user's anxiety about collecting a sample (for example, blood sampling) is lower, the sense of stability is higher. For example, blood sampling by a medical staff provides a high sense of stability to the user. In the degree of restraint, the degree of preference varies depending on the presence or absence of going-out and a waiting time. As going-out is less and a waiting time is shorter, the degree of preference is larger. The hospitals in which the GA test, the 1,5AG test, and the glucose tolerance test can be performed are limited. Thus, in the degree of restraint, the degree of preference is “F”.

In the invasiveness, the degree of preference is larger in a case where a sample can be obtained without pain. In the glucose tolerance test, it is necessary to perform blood sampling three times after glucose tolerance. Thus, the degree of preference for the invasiveness is “F”.

In the required time, as the required time is shorter, the degree of preference is larger. In the risk check for various diseases, the degree of preference is larger in a case where there are many test items and tests for other diseases other than diabetes are included. In the accuracy, as a detection accuracy for postprandial hyperglycemia is higher, the degree of preference is larger.

Further, FIG. 7 illustrates various abnormal tendencies of the blood glucose equivalent value. Specifically, FIG. 7 illustrates an abnormal tendency in which a postprandial hyperglycemic spike is large, an abnormal tendency in which a postprandial hyperglycemic spike is small, an abnormal tendency in which fasting blood glucose is high, and an abnormal tendency in which caution is required for the blood glucose value (caution required for blood glucose value).

Further, in FIG. 7, for each abnormal tendency, a column of the test method to be a test candidate is marked with 0. That is, in a case where a postprandial hyperglycemic spike is large, five types of the test methods (3), (4), (6), (11), and (12) including the test methods (11) and (12) which are specialized for postprandial hyperglycemia and are performed in a hospital are marked with 0. In the test methods (3), (4), and (6), blood sampling after meal is required for determining a postprandial hyperglycemic spike. In a case where the postprandial hyperglycemic spike is small, eight types of the test methods (3) to (10) including the test methods (7) to (9) which are performed at home or at a pharmacy are marked with 0. In a case where fasting blood glucose is high, the test methods (4), (6), and (9) including multiple items are marked with 0. In a case where caution is required for the blood glucose value, the test methods (1) and (2) are marked with 0 by the decision unit 43.

In the present embodiment, the table illustrated in FIG. 7 is stored, as a table, in the storage 13. The decision unit 43 decides a test candidate according to the abnormal tendency of the blood glucose equivalent value determined by the determination unit 42 by referring to the table stored in the storage 13. Specifically, in a case where the abnormal tendency determined by the determination unit 42 corresponds to the abnormal tendency in which a postprandial hyperglycemic spike is large, the decision unit 43 decides the test methods (3), (4), (6), (11), and (12) as test candidates by referring to the table. In a case where the abnormal tendency determined by the determination unit 42 corresponds to the abnormal tendency in which a postprandial hyperglycemic spike is small, the decision unit 43 decides the test methods (3) to (10) as test candidates. In a case where the abnormal tendency determined by the determination unit 42 corresponds to the abnormal tendency in which a fasting blood glucose value is high, the decision unit 43 decides the test methods (4), (6), and (9) as test candidates. In a case where the abnormal tendency determined by the determination unit 42 corresponds to the abnormal tendency in which a blood glucose value is high, the decision unit 43 decides the test methods (1) and (2) as test candidates.

In a case where the user captures an image of a meal using the camera 18 of the mobile terminal 2, the decision unit 43 may determine meal content based on the image of the meal and decide the test candidate according to the meal content. For example, in a case where the abnormal tendency determined by the determination unit 42 corresponds to the abnormal tendency in which a postprandial hyperglycemic spike is small, the decision unit 43 determines meal content. In a case where the meal content is low in glucose, it is considered that a postprandial hyperglycemic spike is small due to an influence of the meal. Thus, the decision unit 43 may change the abnormal tendency of the blood glucose equivalent value to the abnormal tendency in which a postprandial hyperglycemic spike is large, and decide the test methods (3), (4), (6), (11), and (12) as test candidates.

The presentation unit 44 presents, to the user, plural action candidates including the test candidate decided by the decision unit 43. In the present embodiment, the presentation unit 44 presents, to the user, plural action candidates including the test candidate by displaying the test candidate decided by the decision unit 43 on the touch panel 14.

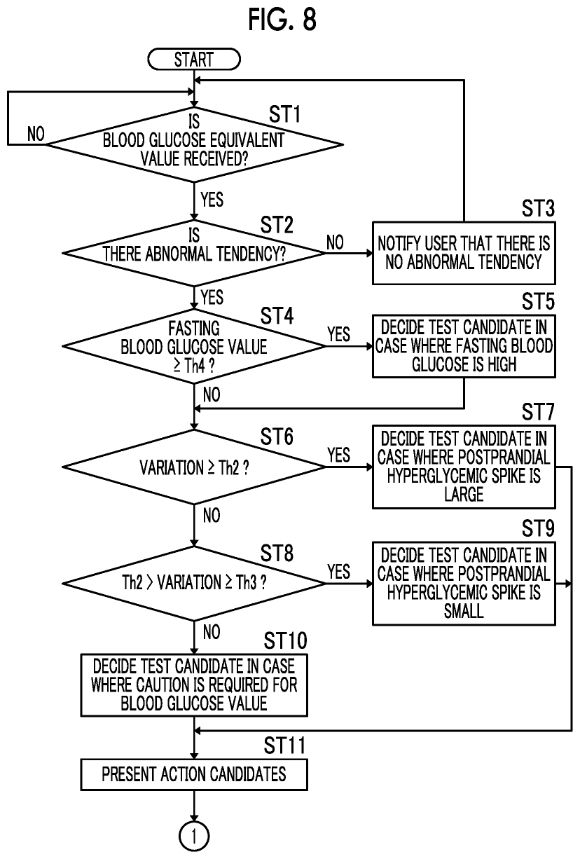

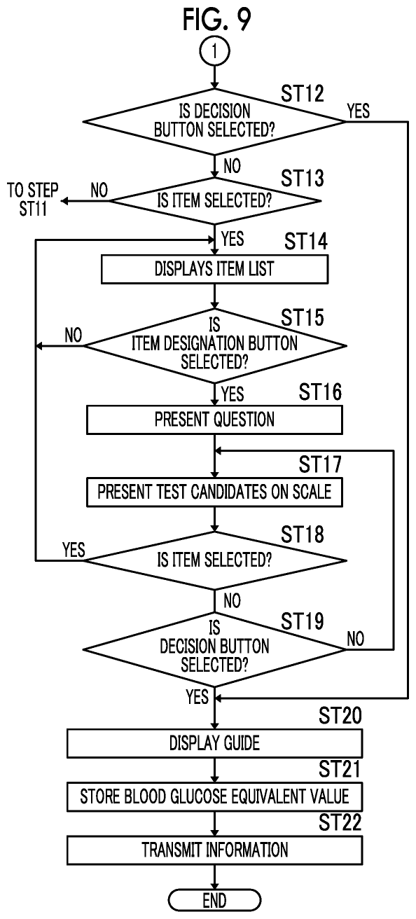

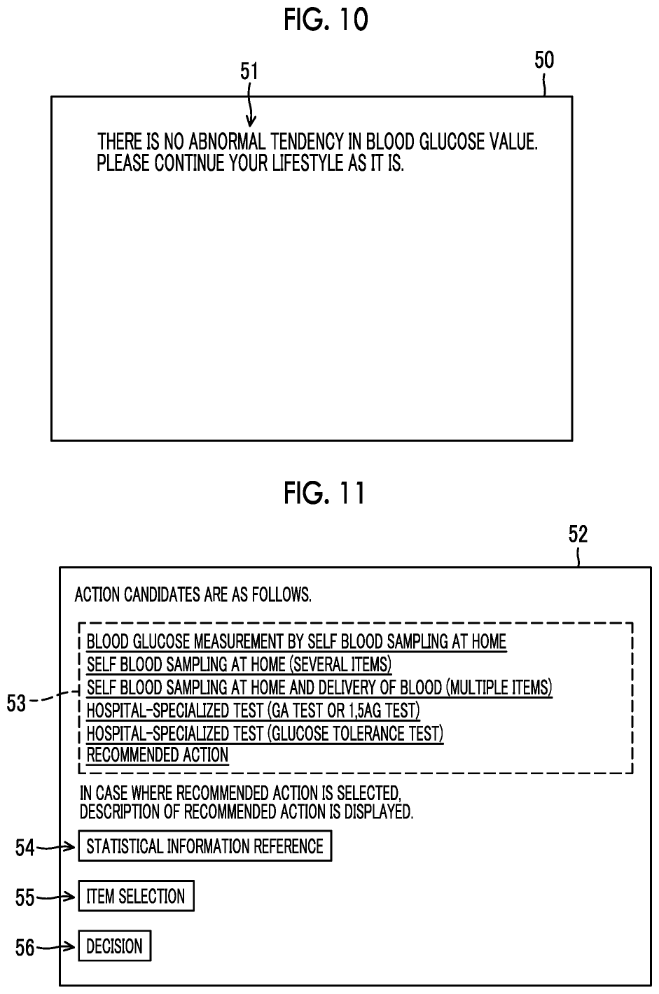

Hereinafter, processing performed in the present embodiment will be described. FIG. 8 and FIG. 9 are flowcharts illustrating processing performed in the present embodiment. In order to monitor the blood glucose equivalent value, the information acquisition unit 41 monitors whether or not the communication I/F 15 receives the blood glucose equivalent value transmitted from the measurement device 3 (step ST1). In a case where a monitoring result in step ST1 is YES, the determination unit 42 determines whether or not there is an abnormal tendency in the blood glucose equivalent value (step ST2). In a case where a determination result in step ST2 is NO, the determination unit 42 notifies the user that there is no abnormal tendency in the blood glucose equivalent value (step ST3), and the process returns to step ST1. FIG. 10 is a diagram illustrating a notification screen in a case where there is no abnormal tendency in the blood glucose equivalent value. As illustrated in FIG. 10, on the notification screen 50, a notification 51 indicating “There is no abnormal tendency in the blood glucose value. Please continue your lifestyle as it is” is displayed.

In a case where a determination result in step ST2 is YES, the determination unit 42 determines a degree of the abnormal tendency of the blood glucose equivalent value. First, the determination unit 42 determines whether or not a fasting blood glucose value of the user is equal to or larger than the threshold value Th4 based on the monitoring result of the blood glucose equivalent value (step ST4). In a case where a determination result in step ST4 is YES, the decision unit 43 decides, as a test candidate, a test method in a case where fasting blood glucose is high (step ST5). In a case where a determination result in step ST4 is NO or after step ST5, the determination unit 42 determines whether or not a variation in the blood glucose equivalent value is equal to or larger than the threshold value Th2 (step ST6).

In a case where a determination result in step ST6 is YES, the decision unit 43 decides, as a test candidate, a test method in a case where a postprandial hyperglycemic spike is large, among the test methods included in the table illustrated in FIG. 7 (step ST7). In a case where a determination result in step ST6 is NO, the determination unit 42 determines whether or not a variation value in the blood glucose equivalent value is equal to or larger than the threshold value Th3 and smaller than the threshold value Th2 (step ST8). In a case where a determination result in step ST8 is YES, the decision unit 43 decides, as a test candidate, a test method in a case where a postprandial hyperglycemic spike is small, among the test methods included in the table illustrated in FIG. 7 (step ST9). In a case where a determination result in step ST8 is NO, the decision unit 43 decides, as a test candidate, a test method in a case where caution is required for the blood glucose value, among the test methods included in the table illustrated in FIG. 7 (step ST10). After step ST7, step ST9, and step ST10, the presentation unit 44 presents, to the user, plural action candidates including the decided test candidate (step ST11).

FIG. 11 is a diagram illustrating an action candidate presentation screen. As illustrated in FIG. 11, on the action candidate presentation screen 52, as an example, the plurality of action candidates 53 including the test candidate in a case where a postprandial hyperglycemic spike is large are displayed. The action candidates 53 include five test candidates and one recommended action. The recommended action represents an action recommended for the user. The five test candidates include blood glucose measurement by self blood sampling at home in the test method (3), self blood sampling at home (several items) in the test method (4), self blood sampling at home and delivery of blood (multiple items) in the test method (6), a hospital-specialized test (GA test or 1,5AG test) in the test method (11), and a hospital-specialized test (glucose tolerance test) in the test method (12). Further, the presentation screen 52 includes a statistical information reference button 54, an item selection button 55, and a decision button 56. The statistical information reference button 54 is a button for displaying statistical information to be described later. The item selection button 55 is a button for displaying an item list screen including an item serving as an index in a case where the user selects a test candidate. The decision button 56 is a button for deciding a test candidate.

Further, the test candidate in a case where the fasting blood glucose value is high, the test candidate in a case where a postprandial hyperglycemic spike is large, the test candidate in a case where a postprandial hyperglycemic spike is small, and the test candidate in a case where caution is required for the blood glucose value may be overlapped. For this reason, for example, in a case where a postprandial hyperglycemic spike is large and the fasting blood glucose value is high, the action candidates 53 include both of the test candidate in a case where a postprandial hyperglycemic spike is large and the test candidate in a case where the fasting blood glucose value is high.

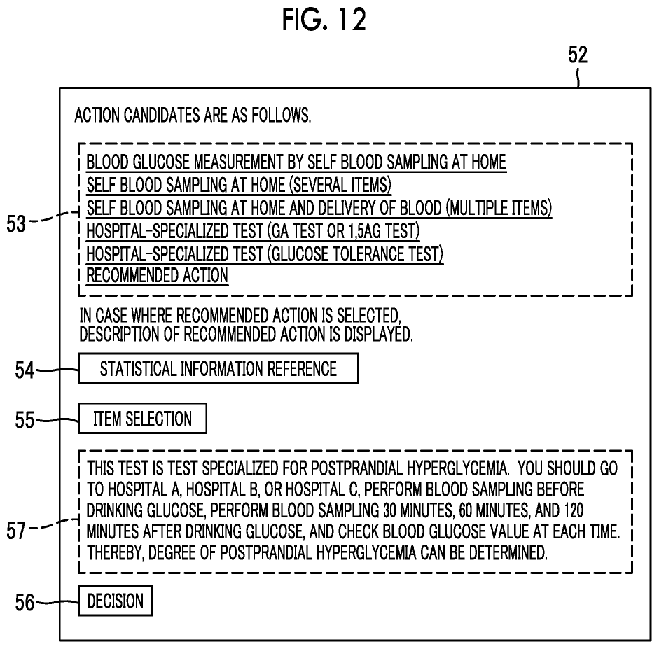

Each test candidate and each recommended action included in the action candidates 53 can be selected, and in a case where the user selects a test candidate or a recommended action included in the action candidates, a description of the selected test candidate or the selected recommended action is displayed. For example, in a case where the user selects “hospital-specialized test (glucose tolerance test)” among the five test candidates, as illustrated in FIG. 12, a description 57 indicating “This test is a test specialized for postprandial hyperglycemia. You should go to hospital A, hospital B, or hospital C, perform blood sampling before drinking glucose, perform blood sampling 30 minutes, 60 minutes, and 120 minutes after drinking glucose, and check a blood glucose value at each time. Thereby, a degree of postprandial hyperglycemia can be determined.” is displayed between the item selection button 55 and the decision button 56. In a case where the user selects the decision button 56 in this state, the selected test candidate is decided as the test method to be performed by the user. Processing after the test method is decided will be described later.

In the test methods (3), (4), and (6), it is necessary to perform blood sampling after meal. Thus, in a case where the test methods (3), (4), and (6) are selected, a description indicating that it is necessary to perform blood sampling after meal is included.

Further, in a case where the user selects the statistical information reference button 54, statistical information of the test candidate which is previously selected is displayed. Here, plural test candidates are selected and presented to the user according to the abnormal tendency of the blood glucose equivalent value of the user, and the user selects one test candidate among the plurality of test candidates and performs the test. The statistical information represents a ratio that each of the plurality of test candidates is previously selected by the user who currently performs a test and/or another user. The statistical information is obtained by counting the test candidates which are previously selected from the test candidates having the same abnormal tendency as the abnormal tendency of the blood glucose equivalent value of the user. For example, in a case where the abnormal tendency of the blood glucose equivalent value of the user is an abnormal tendency in which a postprandial hyperglycemic spike is large, the statistical information for the test methods (3), (4), (6), (11), and (12) is displayed. Further, in a case where the abnormal tendency of the blood glucose equivalent value of the user is an abnormal tendency in which a postprandial hyperglycemic spike is small, the statistical information for the test methods (3) to (10) is displayed.

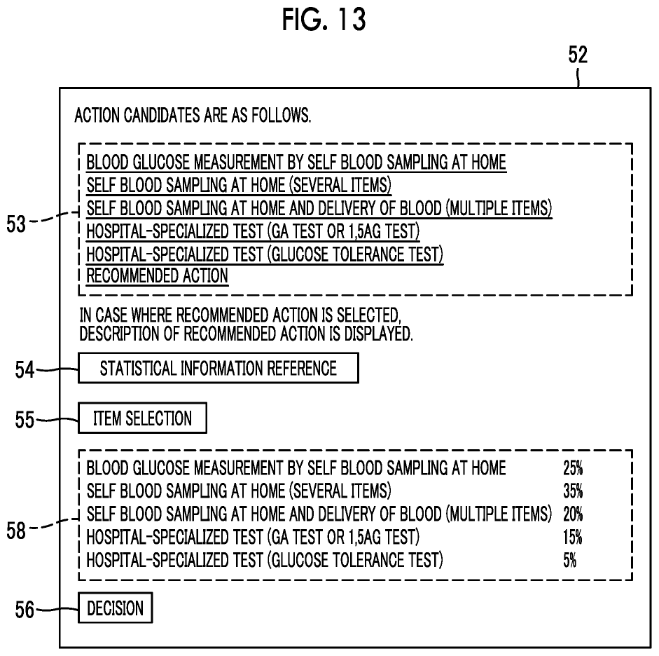

FIG. 13 is a diagram illustrating an action candidate presentation screen on which statistical information of the test candidate is displayed. As illustrated in FIG. 13, statistical information 58 for each of the presented five test candidates is displayed between the item selection button 55 and the decision button 56. As illustrated in FIG. 13, in the statistical information 58, the ratio that each test candidate is previously selected is indicated by a percentage. The statistical information 58 is stored in the test server 6. In a case where the statistical information reference button 54 is selected, the mobile terminal 2 accesses the test server 6, acquires the statistical information 58, and presents the statistical information 58 to the user.

Even in a state where the statistical information 58 is displayed, in a case where the user selects one of the test candidates and selects the decision button 56, the selected test candidate is decided as the test method to be performed by the user.

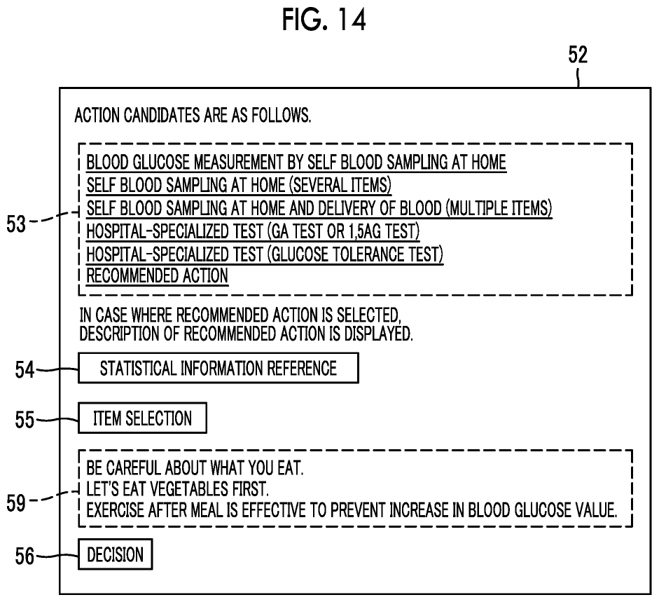

Further, in a case where the user selects a recommended action included in the action candidates 53, the action recommended for the user is displayed according to the blood glucose equivalent value of the user. For example, as illustrated in FIG. 14, a recommended action 59 indicating “Be careful about what you eat. Let's eat vegetables first. Exercise after meal is effective to prevent an increase in blood glucose value.” is displayed between the item selection button 55 and the decision button 56.

After the action candidates are presented, the decision unit 43 determines whether or not the decision button 56 is selected (step ST12), and in a case where a determination result in step ST12 is YES, the process proceeds to step ST20 to be described later. In a case where a determination result in step ST12 is NO, the decision unit 43 determines whether or not the item selection button 55 is selected (item selection, step ST13). In a case where a determination result in step ST13 is NO, the process returns to step ST11. In a case where a determination result in step ST13 is YES, the presentation unit 44 displays an item list (step ST14).

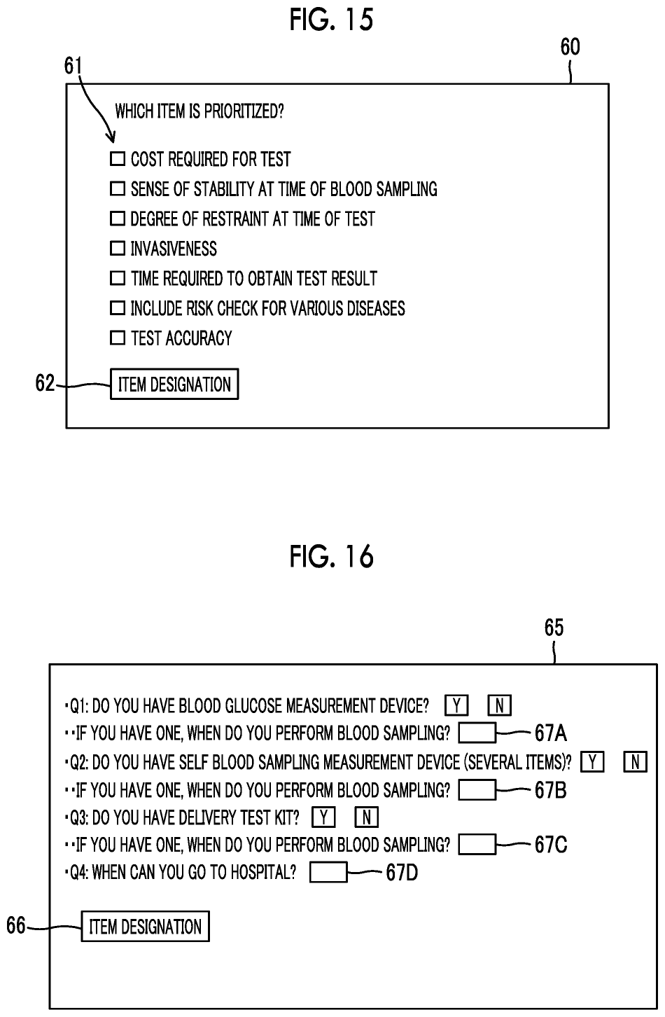

FIG. 15 is a diagram illustrating a list screen for displaying an item list. As illustrated in FIG. 15, a text “which item is prioritized?” is displayed on the item list screen 60. Further, on the item list screen 60, as an item serving as an index in a case where the user selects a test candidate, a cost required for the test, a sense of stability at the time of blood sampling, a degree of restraint at the time of the test, invasiveness or non-invasiveness, a time required to obtain a test result, a risk check for various diseases, and a test accuracy are displayed. Further, a check box 61 is added to each item. The user designates an item by checking the check box of a certain item that the user wants to prioritize in a case of selecting the test information, and selects an item designation button 62. For this reason, the decision unit 43 determines whether or not the item designation button 62 is selected (step ST15). In a case where a determination result in step ST15 is NO, the process returns to step ST14. In a case where a determination result in step ST15 is YES, the decision unit 43 presents, to the user, a question for determining a display position of the test candidate according to the designated item on a scale to be described (step ST16).

FIG. 16 is a diagram illustrating a question presentation screen. For the sake of explanation, it is assumed that the user selects “a required time to obtain a test result” as an item serving as an index in a case of selecting a test candidate. As illustrated in FIG. 16, on the question presentation screen 65, four questions Q1 to Q4 are displayed. The question Q1 is “Do you have a blood glucose measurement device?”, the question Q2 is “Do you have a self blood sampling measurement device (several items)?”, the question Q3 is “Do you have a delivery test kit?”, and the question Q4 is “When can you go to hospital?”. The user answers YES or NO to the questions Q1 to Q3, and in a case where the answer is YES, inputs a blood sampling date and time in input boxes 67A to 67C. For the question Q4, the user inputs, in an input box 67D, a date and time when he/she is likely to go to hospital. Here, it is assumed that the user answers NO to the questions Q1 to Q3 because he/she does not have any of the blood glucose measurement device, the self blood sampling measurement device, and the delivery test kit. For the question Q4, it is assumed that the user inputs a tomorrow's date and time.

In a case where the user inputs an answer and then selects an answer completion button 66, the decision unit 43 decides a position for displaying the test candidate on a scale according to the designated item. The presentation unit 44 presents the test candidates to the user in a display form in which icons representing the test candidates are displayed, at the decided display positions on the scale (presentation of the test candidates on scale, step ST17). Here, in the present embodiment, “a required time to obtain a test result” is selected as an item serving as an index in a case where the user selects a test candidate. Thus, the decision unit 43 obtains a required time to obtain a test result according to the answer to the question, and decides a display position of the test candidate on a scale representing the required time. The presentation unit 44 presents the test candidates to the user in a display form in which test candidate icons are displayed at the decided display positions on the scale.

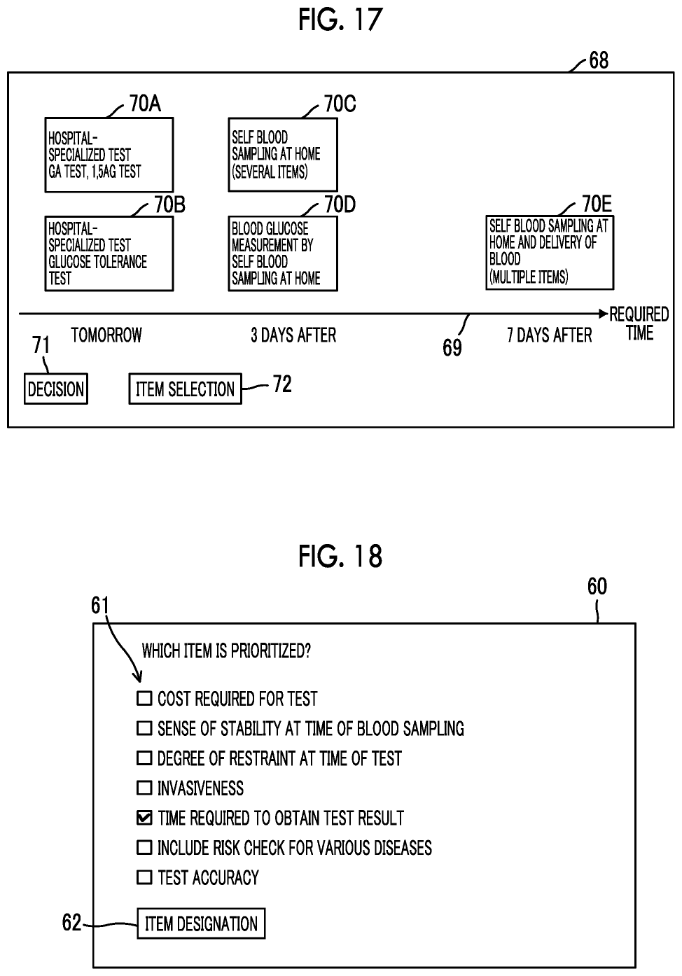

FIG. 17 is a diagram illustrating a presentation screen for presenting the test candidates to the user in a display form in which test candidate icons are displayed on the scale. As illustrated in FIG. 17, on the presentation screen 68, a scale 69 representing the required time is displayed, and five test candidate icons 70A to 70E are displayed so as to be arranged along the scale 69 according to the required time. As illustrated in FIG. 17, the display positions of the test candidate icons 70A and 70B corresponding to the test methods (11) and (12) at a hospital on the scale 69 are “tomorrow”. This is because the user answers that he/she is going to hospital tomorrow in response to the question Q4. In addition, the display positions of the test candidate icon 70C corresponding to the self blood sampling test method (4) at home and the test candidate icon 70D corresponding to the test method (3) on the scale 69 are “3 days after”. “3 days” is a time required to deliver a blood glucose measurement device, a self blood sampling measurement device, and a delivery test kit from the test center to the user. In addition, the display position of the test candidate icon 70E corresponding to the test method (6), which includes self blood sampling at home and delivery of blood, on the scale 69 is “7 days after”. “7 days after” is a time required to deliver a test kit from the test center to the user, deliver a result obtained by performing blood sampling by the user to the test center, perform a test at the test center, and transmit a test result to the mobile terminal 2 of the user.

In a case where the user has a blood glucose measurement device and answers that he/she can immediately perform blood sampling, the required time is, for example, “5 minutes”. Further, in a case where the user has a delivery test kit, the required time is obtained as “4 days after” by subtracting, from 7 days, 3 days which is the number of days required to deliver the test kit to the user.

The user can recognize the required time for the presented test candidate at a glance based on the presentation screen 68. Thereby, it is possible to easily compare the test candidates according to the item that the user prioritizes.

The user can decide a test method desired by the user by selecting a test candidate icon and selecting a decision button 71 on the presentation screen 68. Further, the user can additionally designate an item serving as an index in a case of selecting a test candidate, by selecting an item selection button 72. Thus, after step ST17, the decision unit 43 determines whether or not the item selection button 72 is selected (step ST18). In a case where a determination result in step ST18 is YES, the process returns to step ST14, and processing of step ST14 to step ST18 is repeated. In a case where a determination result in step ST18 is NO, the decision unit 43 determines whether or not the decision button 71 is selected (step ST19). In a case where a determination result in step ST19 is YES, the process proceeds to step ST20 to be described later. In a case where a determination result in step ST19 is NO, the process returns to step ST17.

FIG. 18 is a diagram illustrating an item list screen displayed twice in a case where a determination result in step ST18 is YES. As illustrated in FIG. 18, among the check boxes 61 displayed on the item list screen 60, a check box of “a required time to obtain a test result” is already checked. The user can further select an item on the item list screen 60. Here, it is assumed that the user further selects “a cost required for the test”. The decision unit 43 presents, to the user, a question for deciding the display position of the test candidate on the scale according to “a cost required for the test”.

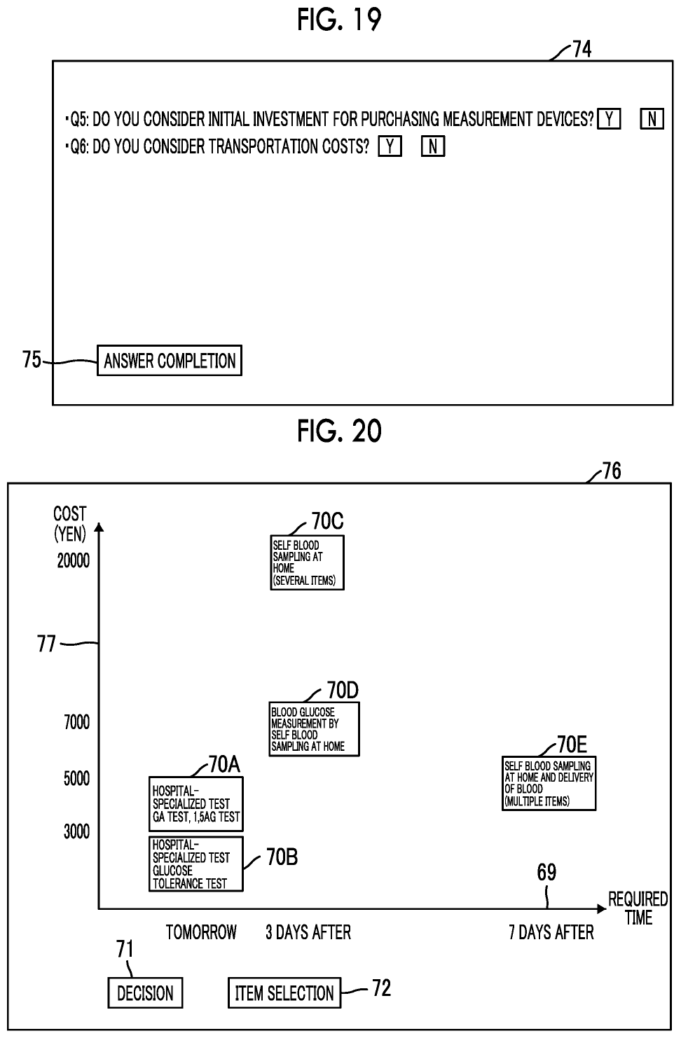

FIG. 19 is a diagram illustrating a question presentation screen related to “a cost required for the test”. As illustrated in FIG. 19, two questions Q5 and Q6 are presented on the question presentation screen 74. The question Q5 is “Do you consider initial investment for purchasing measurement devices?”, and the question Q6 is “Do you consider transportation costs?”. The user answers YES or NO to the question. Here, it is assumed that the user answers YES to all of the questions Q5 and Q6. After the answering, in a case where the user selects the answer completion button 75, the presentation unit 44 updates a display form of the icon representing the test candidate. That is, the decision unit 43 decides a display position of the test candidate on an additional scale according to the additionally-designated item, and the presentation unit 44 presents, to the user, the test candidates at the decided display positions on the presented scale and the additional scale, in a display form in which icons representing the test candidates are displayed.

Specifically, the decision unit 43 obtains a cost required for the test according to the answer to the question, and decides a display position of the test candidate on the scale representing the cost required for the test. The presentation unit 44 presents the test candidates to the user by displaying the scale of the required time and the scale of the cost required for the test such that the two scales are perpendicular to each other and displaying the test candidate icons at the positions on the two scales perpendicular to each other.

FIG. 20 is a diagram illustrating a presentation screen for presenting the test candidates to the user in a display form in which the test candidate icons are displayed on the scale in a case where two items are selected. As illustrated in FIG. 20, on the presentation screen 76, the scale 69 representing the required time and a scale 77 representing the cost are displayed so as to be perpendicular to each other, and five test candidate icons 70A to 70E are displayed so as to be arranged in a two-dimensional shape according to the required time and the cost. As illustrated in FIG. 20, in a case of the test candidate icons 70A and 70B corresponding to the test methods (11) and (12) at a hospital, the display positions on the scale 69 correspond to “tomorrow”, and the display positions on the scale 77 correspond to 3000 yen. Further, in a case of the test candidate icon 70C corresponding to the test method (4) including self blood sampling at home, the display position on the scale 69 corresponds to “3 days after”, and the display position on the scale 77 corresponds to 20000 yen. In a case of the test candidate icon 70D corresponding to the test method (3) including self blood sampling at home, the display position on the scale 69 corresponds to “3 days after”, and the display position on the scale 77 corresponds to 7000 yen. Further, in a case of the test candidate icon 70E corresponding to the test method (6) including self blood sampling at home and delivery of blood, the display position on the scale 69 corresponds to “7 days after”, and the display position on the scale 77 corresponds to 5000 yen.

The user can recognize the required time and the cost for the presented test candidate at a glance based on the presentation screen 76. Thereby, it is possible to easily compare the test candidates in consideration of both the required time and the cost.

The user can decide the test method desired by the user by selecting the test candidate icon and selecting the decision button 71 on the presentation screen 76. Further, the user can additionally designate an item serving as an index in a case of selecting a test candidate, by further selecting the item selection button 72. Thereby, it is possible to compare the test candidates until the user is satisfied by further adding an item.

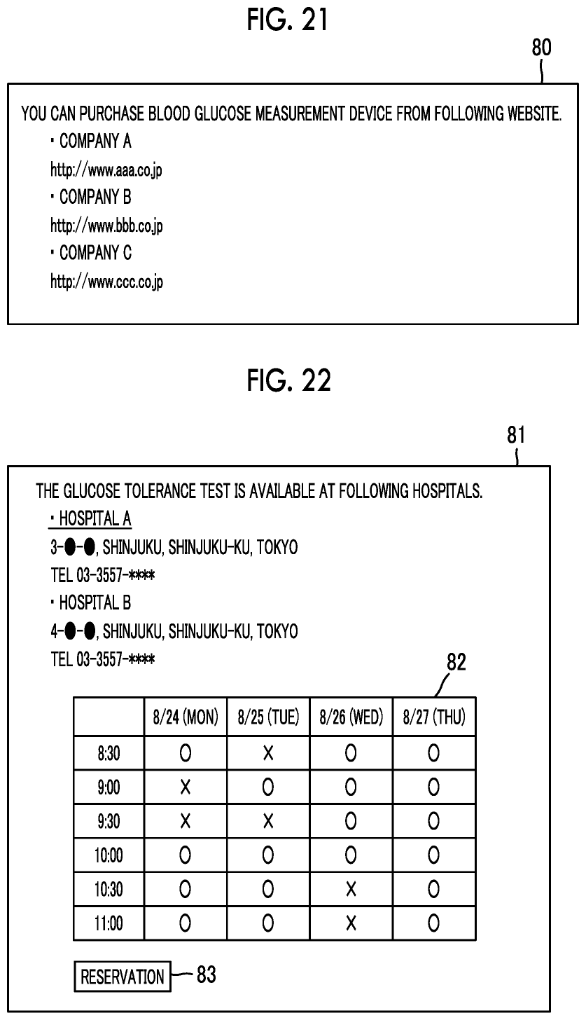

In a case where a determination result in step ST12 is YES, or in a case where a determination result in step ST19 is YES, the presentation unit 44 displays a guide on the test method selected by the user on the touch panel 14 (step ST20). For example, in a case where the user selects a test method including self blood sampling at home and blood glucose measurement because he/she wants to check a blood glucose value on a regular basis in the future, the presentation unit 44 provides, to the user, a guide on a website that sells a blood glucose measurement device (self monitoring of blood glucose (SMBG)). FIG. 21 is a diagram illustrating a guide screen on a website that sells a blood glucose measurement device. As illustrated in FIG. 21, on the guide screen 80, plural websites on which the user can purchase a blood glucose measurement device are displayed. The user can purchase a blood glucose measurement device by accessing the displayed website.

On the other hand, in a case where the user intends to properly check a current state of his/her blood glucose value and selects the glucose tolerance test at hospital, the presentation unit 44 provides, to the user, a guide on a hospital which is located near the user's home or workplace and at which the glucose tolerance test can be performed. FIG. 22 is a diagram illustrating a guide screen for guiding, to the user, a hospital at which the glucose tolerance test can be performed. As illustrated in FIG. 22, on the guide screen 81, a hospital which is located near the user's workplace and at which the glucose tolerance test can be performed is displayed. In addition, a presented hospital name can be selected, and in a case where the hospital name is selected, a list 82 of dates and times for reservation of the glucose tolerance test is displayed. FIG. 22 illustrates a state where a hospital A is selected and a list of dates and times for reservation of the hospital A is displayed. The user can make a reservation for the glucose tolerance test at the selected hospital by selecting the date and time on which “0” is marked and selecting a reservation button 83.

After step ST20, the decision unit 43 stores the acquired blood glucose equivalent value of the user in the storage 13 in association with the acquisition date and time and the abnormal tendency (step ST21). Further, the decision unit 43 associates the acquired blood glucose equivalent value of the user with the acquisition date and time, the abnormal tendency, and the selected test candidate, processes the information such that the user is not specified, and transmits the processed information to the test server 6 (step ST22). Then, the process is completed. In a case where the image of the meal is acquired, the image is also transmitted to the test server 6. In the test server 6, the acquired information is stored.

As described above, in the present embodiment, it is determined whether or not there is an abnormal tendency in the blood glucose equivalent value of the user, and in a case where there is an abnormal tendency in the blood glucose equivalent value of the user, plural action candidates including a test candidate for acquiring biological information associated with the blood glucose equivalent value are presented to the user. Thereby, the user can take an action including performing a test according to the abnormal tendency of his/her blood glucose value. In addition, the user will not perform a test that does not match with the abnormal tendency of his/her blood glucose value. Therefore, it is possible to provide personalized medical care suitable for the user.

In addition, by presenting plural action candidates including a test candidate that can acquire biological information associated with the blood glucose equivalent value with higher accuracy than accuracy of the measurement device 3 or 4, the user can select a test by which his/her blood glucose status can be accurately recognized. Therefore, in a case where the selected test is performed, the user can more accurately recognize his/her blood glucose status.

Further, by receiving designation of at least one item serving as an index in a case where the user selects a test candidate and presenting test candidates in a display form according to the designated item, the test candidates can be presented in a display form which reflects the item that the user wants to prioritize in a case of selecting a test candidate. Therefore, the user can easily select the test candidate.

In addition, by presenting, to the user, a question for determining the display position of the test candidate on the scale according to the designated item, deciding the display position of the test candidate on the scale according to the answer to the question, and presenting, to the user, the test candidate in a display form in which an icon representing the test candidate is displayed at the decided display position on the scale, the test candidate can be presented to the user in a display form which reflects an item serving as an index in a case where the user selects the test candidate. Therefore, the user can select the test candidate in consideration of the item that he/she prioritizes.

Further, by receiving additional designation of at least one item serving as an index in a case where the user selects a test candidate and presenting, to the user, the test candidates by updating the display form of the icons representing the test candidates according to the additionally-designated item, the test candidates can be presented in a display form in which the plurality of items serving as indexes in a case where the user selects a test candidate are considered. Therefore, the user can easily select the test candidate.

Further, by presenting the statistical information of the test candidates which are previously selected to the user, the user can more easily select the test method suitable for himself/herself.

Further, in a case where a blood glucose equivalent value, an abnormal tendency, a selected test candidate, an image of a meal, and the like are stored in the test server 6, the stored information may be used as big data. The stored big data may be used for learning AI that provides information related to blood glucose values, or may be used as statistical information.

In the first embodiment, in a case where the user selects the test method (3), the user possesses a blood glucose measurement device. Therefore, preferably, the mobile terminal 2 periodically notifies the user that it is necessary to measure a blood glucose value.

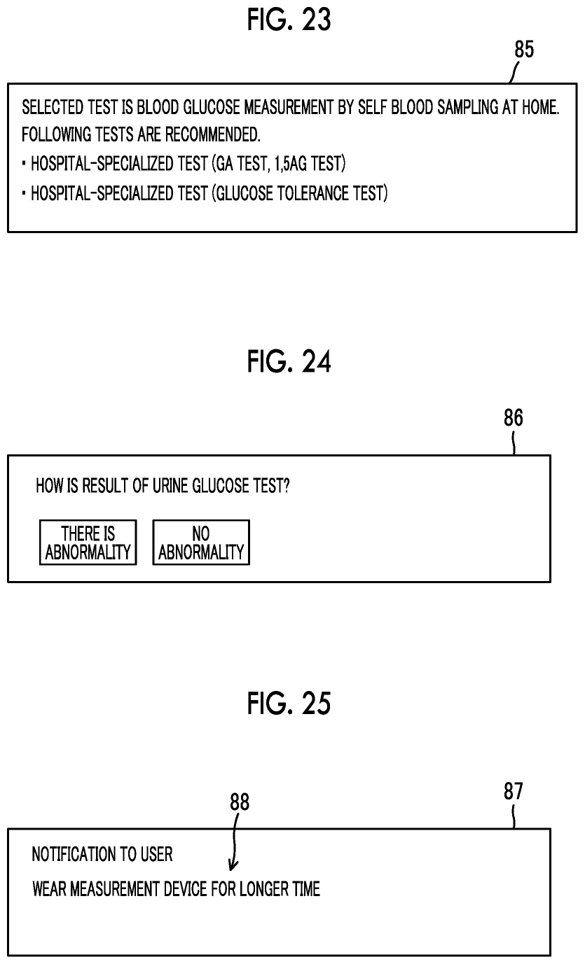

Further, in the first embodiment, in a case where the user selects a test from the test candidates, a guide is provided and the process is completed. On the other hand, in a case where the user selects a test, the action candidates may be suggested as step-up processing. For example, as illustrated in FIG. 11, in a state where the postprandial hyperglycemic spike is large and five test candidates are presented, in a case where the user selects blood glucose measurement including self blood sampling at home, as a next action candidate, the hospital-specialized test (a GA test and a 1,5AG test) of the test method (11) or the hospital-specialized test (a glucose tolerance test) of the test method (12) may be guided. FIG. 23 is a diagram illustrating a guide screen for the next test. As illustrated in FIG. 23, on the guide screen 85, a message indicating that the test selected by the user is blood glucose measurement including self blood sampling at home is displayed, and a message indicating that two test methods of the hospital-specialized (specialized for postprandial hyperglycemia) test (a GA test and a 1,5AG test) and the hospital-specialized test (a glucose tolerance test) are recommended as a next step test is displayed. By referring to the guide screen 85, the user can recognize that it is preferable to perform a hospital-specialized test (a GA test and a 1,5AG test) or a hospital-specialized test (a glucose tolerance test) after the blood glucose measurement including self blood sampling at home.

In the first embodiment, the test method (2) in a case where caution is required for the blood glucose value is a urine glucose test. The urine glucose test is not very accurate as compared to the blood glucose value measured by blood sampling. For this reason, in a state where the urine glucose test is selected by the user and the urine glucose test is performed, in a case where it is determined that the blood glucose value is abnormal, it is preferable to urge the user to measure the blood glucose value by blood sampling. In this case, as illustrated in FIG. 24, an inquiry screen 86 for inquiring of the user about a result of the urine glucose test is displayed. In a state where the user is inquired about the result of the urine glucose test, in a case where non-abnormality is selected, presentation of the action candidates is completed, and in a case where abnormality is selected, as illustrated in FIG. 11, the action candidates in a case where a postprandial hyperglycemic spike is large may be presented to the user.

Further, in the first embodiment, for example, in a case where a measured value of the blood glucose equivalent value is insufficient, whether or not there is an abnormal tendency in the blood glucose value may not be determined. For example, in a case where the user wears the measurement device 3 for a short time, the blood glucose equivalent value may not be monitored enough to determine the postprandial hyperglycemic spike, or the fasting blood glucose equivalent value may not be monitored. In such a case, the determination unit 42 cannot determine whether or not there is an abnormal tendency. Thus, in a case where the determination unit 42 cannot determine whether or not there is an abnormal tendency, preferably, a notification urging the user to wear the measurement device 3 for a longer time is displayed. FIG. 25 is a diagram illustrating a notification screen. As illustrated in FIG. 25, on a notification screen 87, a notification 88 indicating “Please wear the measurement device for a longer time.” is displayed. Based on the notification screen 87, the user can take an action to wear the measurement device 3 for a longer time. Thereby, it is possible to determine whether or not there is an abnormal tendency in the blood glucose equivalent value after the action.

Further, in the first embodiment, on the item list screen 60, every time one item serving as an index in a case where the user selects a test candidate is selected, the test candidates are presented to the user in a display form using a scale. On the other hand, the present disclosure is not limited thereto. On the item list screen 60, the plurality of items may be selected at once. In this case, the display positions of the test candidates on the scale of each of the plurality of selected items are decided, and thus the test candidates are presented to the user in a display form in which the test candidate icons are displayed at the decided display positions.

Further, in the first embodiment, in a case where the item selection button 55 is selected, the item list screen 60 is displayed such that an item serving as an index in a case where the user selects a test candidate can be selected by the user. On the other hand, the present disclosure is not limited thereto. Before monitoring the blood glucose equivalent value by the test support apparatus according to the present embodiment, an item serving as an index in a case where the user selects a test candidate may be selected in advance by the user. In this case, instead of the item selection button 55, a button is displayed on the action candidate presentation screen 52. In a case where the button is selected, the test candidates are presented to the user in the display form illustrated in FIG. 17 or FIG. 20.

In a case where an item serving as an index in a case where the user selects a test candidate is selected in advance by the user, the user may select the item including a risk check for various diseases. In this case, the decision unit 43 may include, in the action candidates, the test candidate including a test with multiple items, which is similar to the test in a case where the fasting blood glucose value is high.

Further, in the first embodiment, in a case where, on the item list screen 60, an item serving as an index in a case where the user selects a test candidate is selected, a question is presented to the user, and the display positions of the test candidates on the scale are decided according to an answer to the question. On the other hand, the present disclosure is not limited thereto. The display positions of the test candidates on the scale may be decided according to a degree of preference of the selected item without a question. For example, in a case where the user selects a sense of stability after blood sampling, the display positions are decided such that a test candidate icon representing blood sampling at a hospital is displayed at a position with a higher sense of stability on the scale representing the sense of stability.

Further, in the first embodiment, on the item list screen 60, items serving as indexes in a case where the user selects the action candidates including not only a test candidate but also a recommended action may be displayed. In this case, the display positions of the action candidates on the scale may be decided according to the selected item, and the action candidate icons may be displayed at the decided display positions.

Further, in the first embodiment, in a case where there is no abnormal tendency in the blood glucose equivalent value, as illustrated in FIG. 10, a notification indicating “There is no abnormal tendency in the blood glucose value. Please continue your lifestyle as it is” is displayed. On the other hand, the content of the notification is not limited thereto. In a case where there is no abnormal tendency in the blood glucose equivalent value but there is an increase tendency in the blood glucose equivalent value, preferably, a notification including an advice for warning the user to be careful about lifestyle is displayed.

Further, in the first embodiment, the blood glucose equivalent value is measured by the measurement devices 3 and 4. On the other hand, the present disclosure is not limited thereto. Instead of measuring the blood glucose equivalent value by the measurement devices 3 and 4, a urine glucose value may be measured by performing a urine glucose test. In this case, the measured urine glucose value is an example of the first biological information. In a case of the urine glucose test, since a urine glucose value is obtained by using a urine glucose test device, the urine glucose value may be used as a blood glucose equivalent value. The urine glucose test device is an example of a measurement device. In this case, the urine glucose test device has a communication function with the mobile terminal 2, and the measured urine glucose value is transmitted to the mobile terminal 2. Thereby, the mobile terminal 2 may monitor the blood glucose equivalent value based on the urine glucose value.

On the other hand, in a case where a urine glucose test is performed by using a test paper, a color of the test paper is changed according to a urine glucose value. In this case, an image of the test paper may be acquired by capturing the test paper by using the camera 18 of the mobile terminal 2, and the determination unit 42 may recognize the color of the test paper from the image of the test paper. Thereby, an abnormal tendency may be determined. In this case, a measurement device is not required, and thus test support can be performed only by the mobile terminal 2. Therefore, the user does not need to prepare the measurement devices 3 and 4 or a urine glucose test device.

Further, the blood glucose equivalent value may be monitored by using an ambulatory glucose profile (AGP) instead of the blood glucose equivalent value measured by the measurement devices 3 and 4. AGP is an analysis method that is useful for reading a tendency of a variation in blood glucose, that is, a blood glucose trend, from blood glucose values for several days obtained by continuous measurement or a glucose value in an interstitial fluid (https://dm-net.co.jp/trend/agp/001.php). By using AGP, it becomes easy to recognize a time zone in which hypoglycemia and hyperglycemia are likely to occur during a day and a time zone in which a variation in the blood glucose value is large. Therefore, by monitoring the blood glucose equivalent value by using AGP, it is possible to easily determine an abnormal tendency of the blood glucose equivalent value.

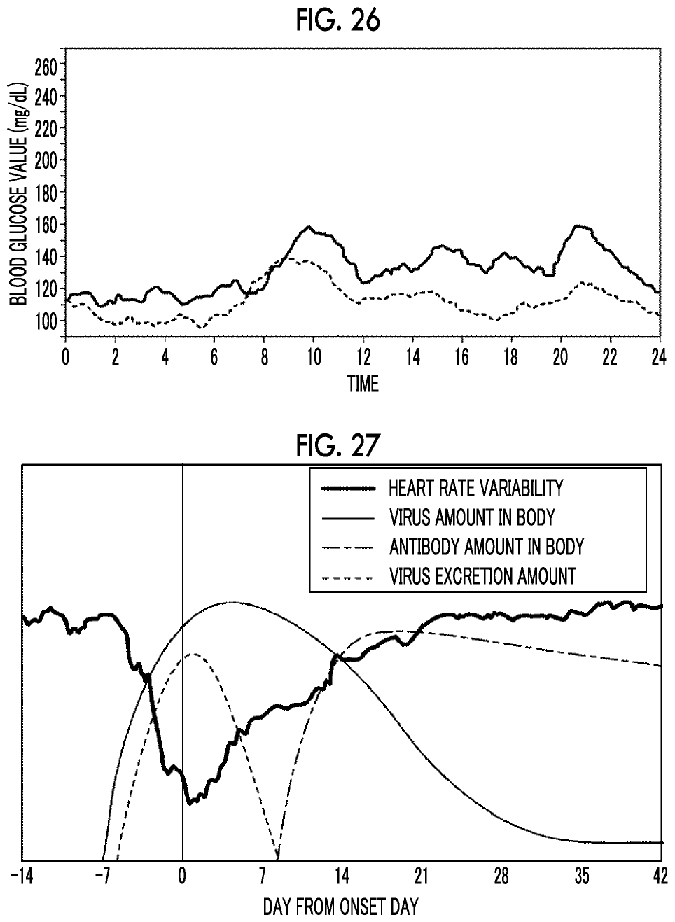

Further, in the first embodiment, the blood glucose equivalent value measured by the measurement devices 3 and 4 is monitored, and thus a comparison result between a monitoring result of the blood glucose equivalent value and a test result obtained by blood sampling of the user may be presented to the user. FIG. 26 is a graph illustrating a comparison result between a monitoring result of the blood glucose equivalent value and a test result obtained by blood sampling of a user. In FIG. 26, a solid line represents a monitoring result of the blood glucose equivalent value, and a broken line represents a test result obtained by blood sampling. As illustrated in FIG. 26, the blood glucose equivalent value measured by the measurement devices 3 and 4 may tend to be higher than the actual blood glucose value. As illustrated in FIG. 26, a comparison result between a monitoring result of the blood glucose equivalent value and a test result obtained by blood sampling of the user is presented to the user. Thereby, the user can determine a deviation between the current blood glucose equivalent value and the actual blood glucose value.

Further, in the first embodiment, a postprandial hyperglycemic spike is used to determine an abnormal tendency in the blood glucose equivalent value. On the other hand, the present disclosure is not limited thereto. As an abnormal tendency determined by monitoring the blood glucose equivalent value, fasting hyperglycemia, hypoglycemia, nocturnal hyperglycemia, or the like may be determined. For example, in a case of hypoglycemia, the test candidates to be presented include a 5-hour tolerance test, a salivary cortisol test, a salivary cortisol+DHEA test, a delayed-type food allergy test, and organic acid urine measurement. The 5-hour tolerance test is an almost essential test as a test for hypoglycemia. The salivary cortisol test is a test to determine whether or not a person is in a fatigued state due to stress. The DHEA test is a test for dehydroepiandrosterone sulfate. The dehydroepiandrosterone sulfate is a type of male hormone. The delayed-type food allergy test is a test to determine severity of intestinal disorders. The organic acid urine measurement is a test to determine whether a fungus grows in an intestinal tract and produces toxins.

### Second Embodiment

In the first embodiment, an example in which the action candidates including the test candidates for acquiring biological information associated with the blood glucose equivalent value according to the abnormal tendency in the blood glucose equivalent value are presented to the user has been described. On the other hand, the present disclosure is not limited thereto. For example, the technique of the present disclosure may be applied to an example in which action candidates including test candidates for acquiring biological information required for diagnosing various diseases such as infectious diseases and cancers are presented to a user. It is known that some of the various diseases cause an abnormal tendency in physiological information such as a heart rate, a blood pressure, respiration, an electrocardiogram, maximum oxygen intake, arterial oxygen saturation, and a body temperature in a case of infection. In particular, in a case where the technique of the present embodiment can be used to determine abnormal tendencies in various physiological information before a user becomes aware of symptoms of the diseases and recommend the user to take various tests in a case where there is an abnormal tendency, it can contribute to early detection of diseases.

Hereinafter, an example in which a new coronavirus infection (COVID-19) is applied as a specific example of the diseases will be described. In the present embodiment, instead of the blood glucose equivalent value in the first embodiment, a heart rate is applied as the first biological information. It is assumed that a heart rate is acquired by a sensor included in the measurement device 3. Further, instead of the glucose, the urine glucose, the blood glucose value, HbA1c, glycoalbumin, and 1,5AG in the first embodiment, a test result related to the presence or absence of infection with new coronavirus is applied as second biological information. In the present embodiment, a repeated description for the same configuration and operation as those in the first embodiment will be omitted.

FIG. 27 is a schematic diagram illustrating an example of changes in heart rate variability (HRV), virus amount in a body, antibody amount in a body, and virus excretion amount of a patient who is infected with new coronavirus infection. In FIG. 27, the heart rate variability is illustrated by a thick solid line, the virus amount in a body is illustrated by a fine solid line, the antibody amount in a body is illustrated by a one-dotted line, and the virus excretion amount is illustrated by a broken line. In a horizontal axis of FIG. 27, an onset day of a disease when a user becomes aware of symptoms such as fever and cough is set as day 0, a day after the onset day of the disease is indicated as a day with plus, and a day before the onset day of the disease is indicated as a day with minus. The heart rate variability is a value representing variability in an R-R interval (RRI) for each heart rate. Specifically, the heart rate variability is represented by a standard deviation of the R-R interval (SDNN) for a predetermined period and/or a root mean square of successive two R-R interval differences (rMSSD).

As illustrated in FIG. 27, in a case of the new coronavirus infection, a virus amount in a body begins to increase rapidly about 5 days before the onset day, and this results in an abnormal tendency such as a decrease in heart rate variability. The reason is as follows. In a case of the heart rate variability of an uninfected person, the heart rate increases during exercise or in a tension state, and the heart rate decreases in a relaxation state. On the other hand, in a case of the heart rate variability of an infected person, followability of the heart rate is poor and a degree of increase/decrease in the heart rate is small. Thereafter, near the onset day, the virus amount in the body reaches a maximum value and the heart rate variability reaches a minimum value. Further, about 10 days after the onset day, the antibody amount in the body begins to increase and the virus amount in the body begins to decrease, and thus the heart rate variability gradually returns to normal.

The information acquisition unit 41 acquires a heart rate transmitted from the communication I/F 25 of the measurement device 3 by receiving the heart rate by the communication I/F 15. Since the heart rate is basically transmitted from the measurement device 3 at all times, that is, at predetermined time intervals, the information acquisition unit 41 constantly acquires the heart rate. The heart rate acquired by the information acquisition unit 41 is stored in the storage 13 in association with the acquisition date and time.

The determination unit 42 determines whether or not there is an abnormal tendency in the heart rate by monitoring the heart rate acquired by the information acquisition unit 41. Specifically, the determination unit 42 monitors the heart rate transmitted from the measurement device 3, calculates heart rate variability from the monitored heart rate, and determines that there is an abnormal tendency in the heart rate in a case where the heart rate variability is equal to or smaller than a predetermined threshold value Th11. Further, the determination unit 42 determines that a degree of the abnormal tendency in the heart rate is large in a case where the heart rate variability is equal to or smaller than a threshold value Th12 (here, Th12<Th11), and determines that a degree of the abnormal tendency in the heart rate is small in a case where the heart rate variability is equal to or smaller than the threshold value Th11 and larger than the threshold value Th12.

Further, the determination unit 42 may determine the abnormal tendency in the heart rate by using a representative value such as an average value and a median value of the heart rate variability for each predetermined period (for example, 6 hours). This is because it is preferable that a temporary increase in the heart rate variability during exercise and in a tension state is not considered as an abnormal tendency.

Further, in a case where the heart rate variability is in an increase state (equal to or larger than the threshold value Th11), the determination unit 42 determines whether or not the increase is a temporary increase during exercise, in a tension state, or the like. In a case where it is determined that the increase is a temporary increase, the determination unit 42 may determine whether or not there is an abnormal tendency in the heart rate during a period other than a period for which the heart rate variability increases. Whether or not the heart rate variability is a temporary increase may be determined by comparing the heart rate variability with a temporary increase tendency of the heart rate variability in the previous same situation. Specifically, for example, a temporary increase pattern of the heart rate variability is stored in advance in the storage 13, and the determination unit 42 may determine whether or not the heart rate variability during monitoring is a temporary increase by comparing the stored increase pattern with the heart rate variability during monitoring.

In a case where the determination unit 42 determines that there is an abnormal tendency in the heart rate, the decision unit 43 decides plural action candidates to be recommended for the user. The action candidates include at least one test candidate for acquiring a test result related to the presence or absence of infection with new coronavirus.

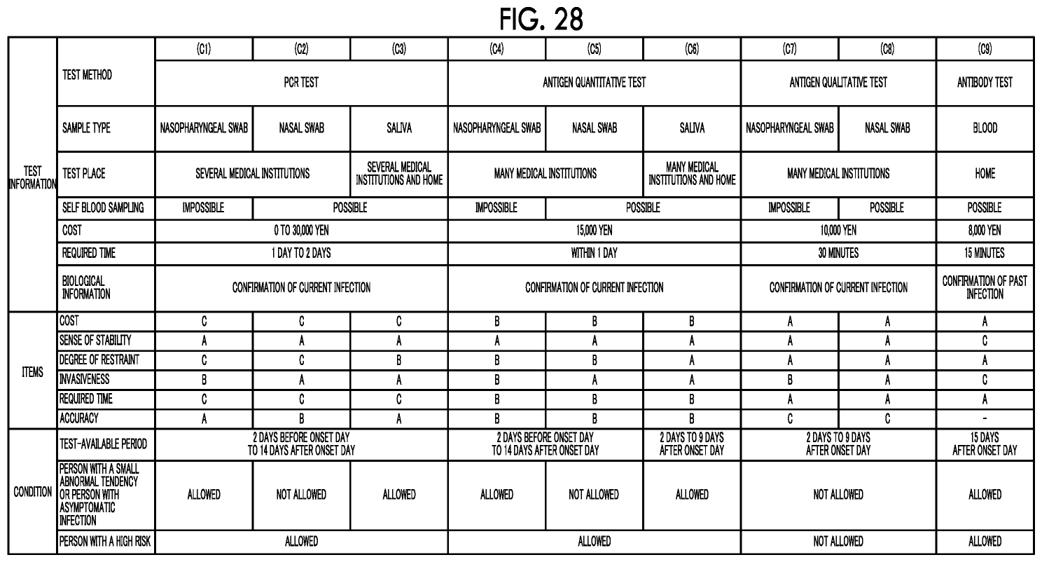

A specific example of a method of deciding a test candidate by the decision unit 43 will be described with reference to FIG. 28. FIG. 28 is a table illustrating test methods related to the new coronavirus infection. In FIG. 28, for 9-type test methods (C1) to (C9), a sample type, a test place, self-collecting availability, a cost, a required time, and biological information to be acquired are illustrated as test information. The table illustrated in FIG. 28 is stored, as a table, for example, in the storage 13. The content of FIG. 28 is an example, and the test information, an evaluation and a condition of each item may be updated as appropriate according to an epidemic situation and a treatment situation of the new coronavirus infection, a progress state of the test method, and the like.

The test methods (C1) to (C3) are polymerase chain reaction (PCR) tests in which the samples are nasopharyngeal swab, nasal swab, and saliva. The test methods (C4) to (C6) are antigen quantitative tests in which the samples are nasopharyngeal swab, nasal swab, and saliva. The test methods (C7) and (C8) are antigen qualitative tests in which the samples are nasopharyngeal swab and nasal swab. In the test methods (C1) to (C8), a test result as to whether or not the patient is currently infected with the new coronavirus infection can be acquired as the second biological information. The test method (C9) is an antibody test using blood as a sample. In the test method (C9), a test result as to whether or not the patient is previously infected with the new coronavirus infection can be acquired as the second biological information. The decision unit 43 selectively decides the test candidate from plural the test methods (C1) to (C9) including designation of a type of the sample and used for acquiring the second biological information from the sample.

FIG. 28 illustrates various items serving as indexes in a case where the user selects the presented test candidate. The definitions of the various items and the evaluation methods are the same as those in the first embodiment, and thus a description thereof will be omitted. On the other hand, in a case of accuracy, as a degree to which a disease-positive person can be detected as positive (so-called sensitivity) is higher, a degree of the accuracy is higher.

FIG. 28 illustrates various conditions in a case where the decision unit 43 decides the test candidate. Specifically, conditions related to a test-available period and test suitability for a person with a small abnormal tendency, a person with asymptomatic infection, and a person with a high risk are illustrated. The “test-available period” indicates a period for which a degree to which a disease-positive person can be detected as positive (so-called sensitivity) in a case where a test is performed for the disease-positive person is relatively high and a test result is reliable. The “person with a small abnormal tendency” is a person determined by the determination unit 42 as having a small abnormal tendency. The “person with asymptomatic infection” means a person whose symptoms such as fever and cough are not developed. For the person with a small abnormal tendency and the person with asymptomatic infection, it is considered that a highly-sensitive test method should be adopted in order to prevent an error in determination due to false negatives, and as a result, a test method with a relatively low sensitivity is not allowed. The “person with a high risk” is a person who has a risk factor such as a serious condition and a high treatment difficulty, and is, for example, an elderly person, a person having an underlying disease, a pregnant woman, or the like. For the person with a high risk, it is considered that a test should be performed with higher accuracy in order to quickly perform a proper treatment, and as a result, a test method with a relatively low sensitivity is not allowed.

The decision unit 43 decides, as a test candidate, a test method suitable for various conditions among the test methods (C1) to (C9) by referring to the table of FIG. 28. As illustrated in FIG. 28, in a case of new coronavirus infection, the test-available period is determined based on the onset day. Firstly, the decision unit 43 estimates the onset day based on the heart rate variability, and decides the test candidate according to whether the current time is included in the test-available period based on the estimated onset day. For example, in a case where it is estimated that the current time is two days before the onset day, the decision unit 43 decides the test methods (C1) to (C5) as test candidates. In addition, for example, in a case where it is estimated that the current time is four days after the onset day, the decision unit 43 decides the test methods (C1) to (C8) as test candidates. Further, for example, in a case where it is estimated that the current time is 15 days or more after the onset day, the decision unit 43 decides the test method (C9) as a test candidate.

As a method for estimating the onset day, based on, for example, a fact that there is a time lag of about 5 days from a time when the abnormal tendency in the heart rate variability starts to be observed to the onset day (refer to FIG. 27), a method of estimating the onset day to about 5 days after a time when the determination unit 42 determines that there is an abnormal tendency in the heart rate may be used. Further, for example, a method of estimating the onset day using a learning model may be used, the learning model being learned by using, as learning data, a pair of transition data of the heart rate variability of a patient who is infected with new coronavirus infection and an actual onset day, and being a model that receives the transition data of the heart rate variability and outputs the estimated onset day.

Secondly, the decision unit 43 decides the test candidate according to the degree of the abnormal tendency in the heart rate that is determined by the determination unit 42. For example, in a case where the determination unit 42 determines that the abnormal tendency is small, the decision unit 43 decides the test methods (C1), (C3), (C4), (C6), and (C9) as test candidates. Further, the decision unit 43 decides the test candidates in the same manner even in a case where the user is a person with asymptomatic infection. On the other hand, in a case where the determination unit 42 determines that the abnormal tendency is large, the decision unit 43 decides the test methods (C1) to (C9) as test candidates.

Thirdly, the decision unit 43 decides the test candidate according to a risk factor that the user has. For example, in a case where the user is a person with a high risk, the decision unit 43 decides the test methods (C1) to (C6) and (C9) as test candidates. On the other hand, in a case where the user is not a person with a high risk, the decision unit 43 decides the test methods (C1) to (C9) as test candidates.

According to the first to third decisions, the decision unit 43 decides a final test candidate. For example, in a case where, the current time is four days after the onset day, the determination unit 42 determines that the abnormal tendency is large, and the user is a person with symptomatic infection and is not a person with a high risk, the decision unit 43 decides the test methods (C1) to (C8) as test candidates. Further, for example, in a case where, the current time is four days after the onset day, the determination unit 42 determines that the abnormal tendency is large, and the user is a person with asymptomatic infection and is a person with a high risk, the decision unit 43 decides the test methods (C1), (C3), (C4), and (C6) as test candidates.

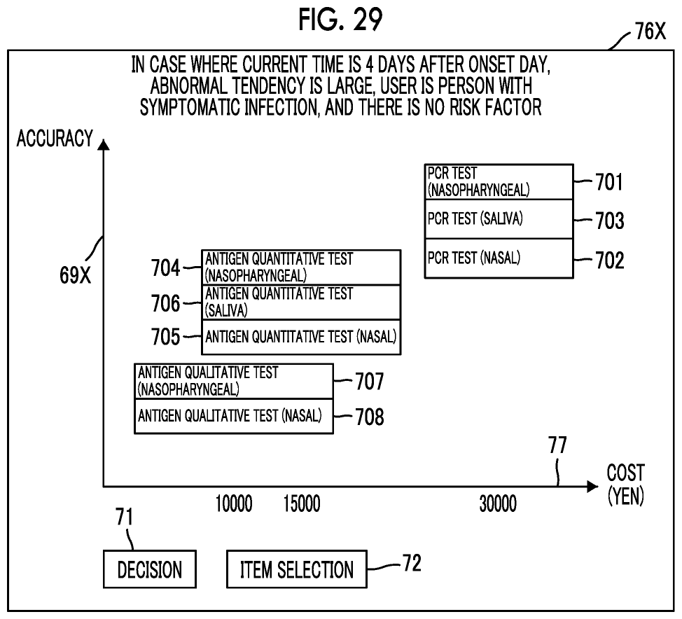

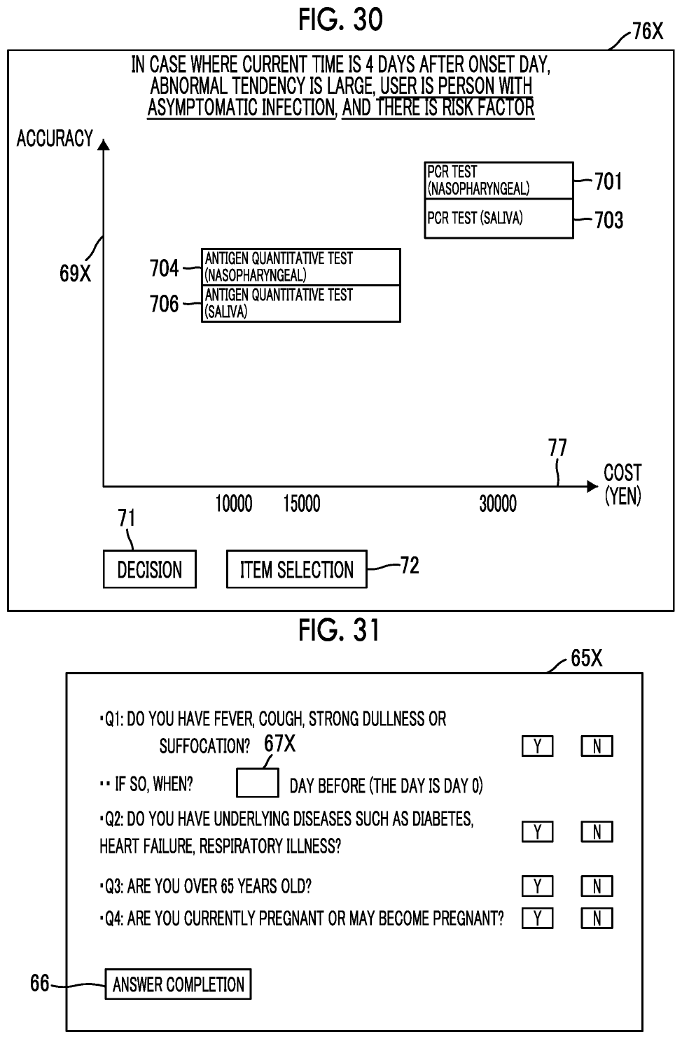

The presentation unit 44 presents, to the user, plural action candidates including the test candidate decided by the decision unit 43. FIG. 29 and FIG. 30 are diagrams illustrating an example of a presentation screen for presenting the test candidates to the user in a display form in which the test candidate icons are displayed on the scale in a case where two items of “cost” and “accuracy” are selected. As illustrated in FIG. 29 and FIG. 30, on the presentation screen 76X, a scale 77 representing the cost and a scale 69X representing the accuracy are displayed so as to be perpendicular to each other, and test candidate icons 701 to 708 are displayed so as to be arranged in a two-dimensional shape according to the cost and the accuracy. The icons 701 to 708 respectively correspond to the test methods (C1) to (C8).

FIG. 29 illustrates a presentation screen in a case where, the current time is four days after the onset day, the determination unit 42 determines that the abnormal tendency is large, and the user is a person with symptomatic infection and is not a person with a high risk. FIG. 30 is a presentation screen in a case where, the current time is four days after the onset day, the determination unit 42 determines that the abnormal tendency is large, and the user is a person with asymptomatic infection and is a person with a high risk. The user can recognize the cost and the accuracy for the presented test candidate at a glance based on the presentation screen 76X. Thereby, it is possible to easily compare the test candidates in consideration of both the cost and the accuracy.

Further, in a state where the current time is three days before the onset day, in a case where the decision unit 43 decides that any of the test methods is not suitable, the presentation unit 44 may present, as an action candidate, a notification indicating a fact that any of the test methods is not suitable. For example, a notification indicating “There is an abnormal tendency in the heart rate. But, at this time, an accurate result cannot be obtained by a test. Please continue to monitor your heart rate.” is presented. Further, in a case where the determination unit 42 determines that there is no abnormal tendency in the heart rate, the presentation unit 44 may present, as an action candidate, a notification indicating a fact that there is no abnormal tendency in the heart rate. For example, a notification indicating “Currently, there is no abnormal tendency in the heart rate. Please continue to take measures against infection.” is presented.

As described above, in the present embodiment, it is determined whether or not there is an abnormal tendency in the heart rate of the user, and in a case where there is an abnormal tendency in the heart rate of the user, plural action candidates including a test candidate for acquiring a test result, which is related to the presence or absence of infection with new coronavirus infection associated with the heart rate, are presented to the user. Thereby, the user can take an action including performing a test according to the abnormal tendency of his/her heart rate. In particular, in a case of new coronavirus infection, the virus excretion amount of a patient who is infected with new coronavirus infection, that is, infectivity to others reaches a peak near the onset day (refer to FIG. 27). Thus, by determining the abnormal tendency in the heart rate before the onset day and presenting the user to take a test, it is possible to contribute to prevention of spread of infection.

In the second embodiment, an example in which the determination unit 42 determines the abnormal tendency in the heart rate based on the heart rate variability has been described. On the other hand, the present disclosure is not limited thereto. The determination unit 42 may determine that there is an abnormal tendency in the heart rate in a case where, instead of the heart rate variability, the heart rate value is equal to or higher than a predetermined threshold value for a predetermined period (for example, three days). According to the example, it is possible to contribute to early detection of a disease in which the heart rate is maintained in a high state at the time of infection. The determination unit 42 may determine that there is an abnormal tendency in the heart rate in a case where, instead of the heart rate variability, a temporal variation in the heart rate value (a difference between a maximum heart rate and a minimum heart rate for a predetermined period (for example, 3 days)) is equal to or larger than a predetermined threshold value. According to the example, it can contribute to early detection of a disease in which the heart rate sharply increases at the time of infection.

Further, in the second embodiment, an example in which the heart rate is applied as the first biological information has been described. On the other hand, the present disclosure is not limited thereto. In the second embodiment, as the first biological information, at least one piece of physiological information such as a heart rate, a blood pressure, respiration, an electrocardiogram, maximum oxygen intake, arterial oxygen saturation, and a body temperature may be applied. The physiological information is acquired by, for example, a sensor included in a wearable terminal such as a smart watch.

Further, in the second embodiment, an example in which a test result related to the presence or absence of infection with new coronavirus infection is applied as the second biological information has been described. On the other hand, the present disclosure is not limited thereto. In the second embodiment, a diagnosis result of a disease of the user may be applied as the second biological information associated with physiological information. As the “diagnosis result of a disease”, for example, an analysis result of components and a detection result of pathogens such as a virus and a bacteria using, as a sample, body fluids such as blood, urine, feces, nasopharyngeal swab, nasal swab, and saliva of the user may be applied. Further, for example, a reading result of an image, which is obtained by imaging an organ as a sample such as a stomach, a large intestine, a lung, a uterus, and a breast of the user by using a method such as computed tomography (CT), magnetic resonance imaging (MM), and ultrasound, may be applied.

Further, in the second embodiment, diagnosis results of plural different types of diseases may be applied as the second biological information associated with physiological information. For example, both of a test related to new coronavirus infection and a test related to influenza virus infection may be presented as test candidates. In addition, an action candidate indicating that the user can take tests related to the plurality of different types of diseases at the same time may be presented. In such a case, diagnosis for various diseases can be recommended, and thus it is possible to contribute to early detection of diseases.

Further, in the second embodiment, the onset day and information on whether the user corresponds to a person with asymptomatic infection or a person with a high risk may be input from the user via, for example, the touch panel 14. FIG. 31 is a diagram illustrating a question presentation screen for inputting an onset day and information on a user. As illustrated in FIG. 31, four questions Q1 to Q4 are displayed on the question presentation screen 65X. The user answers YES or NO to the questions Q1 to Q4. In a case where an answer to the question Q1 is YES, the user inputs an onset day in an input box 67X. Thereby, the decision unit 43 specifies the onset day. In a case where an answer to the question Q1 is NO, the decision unit 43 specifies that the user is a person with asymptomatic infection. In a case where an answer to at least one of the questions Q2 to Q4 is YES, the decision unit 43 specifies that the user is a person with a high risk. In a case where the user selects an answer completion button 66 after answering, the decision unit 43 decides the test candidate according to answer results of the four questions Q1 to Q4.

Further, in the second embodiment, based on a fact that there is a time lag of about 5 days from a time when the abnormal tendency in the heart rate starts to be observed to the onset day (refer to FIG. 27), the decision unit 43 may estimate whether or not the user is a person with asymptomatic infection. For example, in a case where the current time is two days after a time when the abnormal tendency in the heart rate starts to be observed, the decision unit 43 may estimate that the user is a person with asymptomatic infection whose symptoms are not yet developed. Further, for example, in a case where a body temperature is measured by the measurement device 3, the decision unit 43 may determine the presence or absence of fever, and in a case where there is no fever, may estimate that the user is a person with asymptomatic infection.

Further, in the second embodiment, an example in which the decision unit 43 estimates the onset day based on a fact that the test-available period of new coronavirus infection is determined based on the onset day has been described. On the other hand, the present disclosure is not limited thereto. For example, in a case where influenza virus infection is tested, a time from an infection to an onset is as short as 1 day to 2 days, and as a result, a difference between the day when the abnormal tendency in the first biological information starts to be observed and the onset day is small. Thus, significance of estimating the onset day is small. Further, for example, in a case where various lifestyle-related diseases are tested, it is difficult to estimate an accurate onset day. On the other hand, even in these diseases, appropriate test methods may differ according to an elapsed time from a time when the abnormal tendency starts to be observed. The decision unit 43 may decide the test candidate according to an elapsed time from a time when it is determined that there is the abnormal tendency in the first biological information to a current time.

Further, in the second embodiment, as a condition for determining the test candidate, a condition as to whether or not the user is a close contact person may be applied. In a case where the user is a close contact person, possibility that the user is detected as a disease-positive person is relatively high. Thus, it is considered that a highly-sensitive test method should be adopted in order to prevent an error in determination due to false negatives. Therefore, a test method having a relatively low sensitivity may not be allowed. Whether or not the user is a close contact person may be detected by, for example, a known application for new coronavirus contact confirmation.

Further, in the second embodiment, after a test related to the presence or absence of infection with the new coronavirus infection is performed, the test result is fed-back to the mobile terminal 2. In this case, the mobile terminal 2 may determine whether to continue monitoring of the heart rate. For example, in a case where the determination unit 42 determines that there is an abnormal tendency in the heart rate, a test for new coronavirus infection is performed. On the other hand, in spite of the test, in a case where the test result is negative, it may be determined that monitoring of the heart rate is continued. In particular, in a case where the user corresponds to any one of a person with a small abnormal tendency, a person with asymptomatic infection, a person with a high risk, or a close contact person, it is considered that there is a high risk of an error in determination and a sudden change in physical condition due to false negatives. Thus, preferably, it is determined that monitoring of the heart rate is continued.

Specifically, the information acquisition unit 41 acquires a test result related to the presence or absence of infection with new coronavirus infection. The test result may be transmitted from a test agency to the mobile terminal 2, or may be input from the user via the touch panel 14. In a case where the acquired test result is negative, the presentation unit 44 determines that monitoring of the heart rate is continued, and presents a notification indicating a fact that monitoring of the heart rate is continued to the user. In a case where monitoring of the heart rate is continued, the determination unit 42 determines whether or not the abnormal tendency in the heart rate becomes more remarkable and whether or not the abnormal tendency in the heart rate continues. In a case where the determination unit 42 determines that the abnormal tendency in the heart rate becomes more remarkable or that the abnormal tendency in the heart rate continues, the decision unit 43 decides plural action candidates to be recommended again for the user.

Further, in the second embodiment, the presentation unit 44 may transmit heart rate data of the user and the test candidate decided by the decision unit 43 to a test agency to which the user applies for the test. According to the example, it is possible to assist a medical staff in the test agency in determining whether the test method selected by the user is appropriate.

Further, in the embodiment, the presentation unit 44 presents the action candidates to the user by displaying the action candidates on the touch panel 14. On the other hand, the present disclosure is not limited thereto. The presentation unit 44 may present the action candidates to the user by voice.

Further, in the embodiment, in a case where the user takes a medical examination, the test support apparatus according to the embodiment may be used. For example, the measurement devices 3 and 4 may be delivered to the user about two weeks before the medical examination, and thus monitoring of the first biological information may be performed. In a case where an abnormal tendency is observed in the first biological information, an action candidate may be presented to the user, and a test candidate may be selected by the user. Thus, a selected test may be performed at the time of a medical examination.

Further, in the embodiment, the first biological information measured by the measurement devices 3 and 4 is transmitted to the mobile terminal 2, and the mobile terminal 2 presents the action candidates to the user. On the other hand, the present disclosure is not limited thereto. In particular, in a case where the test support program according to the present embodiment is installed in the wristwatch-type measurement device 3, the measurement device 3 may solely determine the abnormal tendency of the first biological information, decide the action candidates, and present the action candidates. In this case, the measurement device 3 has a functional configuration of the test support apparatus according to the present embodiment illustrated in FIG. 5.

Further, in the embodiments, the first biological information measured by the measurement devices 3 and 4 is transmitted to the mobile terminal 2, and the mobile terminal 2 determines whether or not there is an abnormal tendency in the blood glucose equivalent value, determines an action candidate, and presents the action candidate. On the other hand, the present disclosure is not limited thereto. The first biological information measured by the measurement devices 3 and 4 may be transmitted to the test server 6 directly from the measurement devices 3 and 4 or via the mobile terminal 2, and the test server 6 may determine the abnormal tendency, decide the action candidates, and present the action candidates. In this case, the test server 6 transmits the decided action candidates to the mobile terminal 2 or the measurement device 3, and thus the mobile terminal 2 or the measurement device 3 presents the action candidates to the user.

Further, in the embodiments, for example, as a hardware structure of processing units that execute various processing, such as the information acquisition unit 41, the determination unit 42, the decision unit 43, and the presentation unit 44, the following various processors are may be used. The various processors include, as described above, a CPU, which is a general-purpose processor that functions as various processing units by executing software (program), and a dedicated electric circuit, which is a processor having a circuit configuration specifically designed to execute a specific processing, such as a programmable logic device (PLD) or an application specific integrated circuit (ASIC) that is a processor of which the circuit configuration may be changed after manufacturing such as a field programmable gate array (FPGA).

One processing unit may be configured by one of these various processors, or may be configured by a combination of two or more processors having the same type or different types (for example, a combination of plural FPGAs or a combination of a CPU and an FPGA). Further, the plurality of processing units may be configured by one processor.

As an example in which the plurality of processing units are configured by one processor, firstly, as represented by a computer such as a client and a server, a form in which one processor is configured by a combination of one or more CPUs and software and the processor functions as the plurality of processing units may be adopted. Secondly, as represented by a system on chip (SoC) or the like, a form in which a processor that realizes the function of the entire system including the plurality of processing units by one integrated circuit (IC) chip is used may be adopted. As described above, the various processing units are configured by using one or more various processors as a hardware structure.

Further, as the hardware structure of the various processors, more specifically, an electric circuit (circuitry) in which circuit elements such as semiconductor elements are combined may be used.

What is claimed is:

1. A test support apparatus comprising at least one processor, wherein the processor is configured to
determine whether or not there is an abnormal tendency in first biological information of a user by monitoring the first biological information,
determine, as a plurality of action candidates to be recommended for the user, a plurality of action candidates including at least one test candidate for acquiring second biological information associated with the first biological information in a case where there is the abnormal tendency in the first biological information, and
present the plurality of action candidates to the user.

2. The test support apparatus according to claim 1, wherein the action candidates includes the test candidate in which the second biological information is acquired with accuracy higher than accuracy of the first biological information.

3. The test support apparatus according to claim 1, wherein the processor is configured to
receive designation of at least one item serving as an index in a case where the user selects the presented action candidates, and
present the action candidates in a display form according to the designated item.

4. The test support apparatus according to claim 3, wherein the item serves as an index in a case where the user selects the test candidate included in the presented action candidates, and
the processor is configured to
present the test candidate in a display form according to the designated item.

5. The test support apparatus according to claim 4, wherein the processor is configured to
present, to the user, a question for determining a display position of the test candidate on at least one scale according to the designated at least one item,
determine the display position of the test candidate on the scale according to an answer to the question, and
present, to the user, the test candidate in a display form in which an icon representing the test candidate is displayed at the determined display position on the scale.

6. The test support apparatus according to claim 5, wherein the processor is configured to
receive additional designation of the item by the user after the icon representing the test candidate is displayed, and
update the display form of the icon representing the test candidate in a case where designation of the item is added.

7. The test support apparatus according to claim 6, wherein the processor is configured to
repeatedly perform receiving of additional designation of the item, presenting of a question according to the added item, and updating of the display form of the test candidate until the test candidate is selected by the user.

8. The test support apparatus according to claim 4, wherein the item includes at least one of a cost required for a test, a sense of stability in a case where a sample is collected, a degree of restraint in a test, invasiveness or non-invasiveness, a time required to obtain a test result, whether to include a risk check for various diseases, or test accuracy.

9. The test support apparatus according to claim 1, wherein the processor is configured to
selectively determine the test candidate from a plurality of test methods including designation of a type of a sample and used for acquiring the second biological information from the sample.

10. The test support apparatus according to claim 1, wherein the processor is configured to
determine the test candidate according to a degree of the abnormal tendency.

11. The test support apparatus according to claim 1, wherein the processor is configured to
determine the test candidate according to an elapsed time from a time when it is determined that there is the abnormal tendency in the first biological information to a current time.

12. The test support apparatus according to claim 1, wherein the processor is configured to
determine the test candidate according to a risk factor that the user has.

13. The test support apparatus according to claim 1, wherein the processor is configured to
present, to the user, statistical information of the test candidate which is previously selected.

14. The test support apparatus according to claim 1, wherein
the first biological information is a blood glucose equivalent value that correlates with a blood glucose value, and
the second biological information includes at least one of a blood glucose value or HbA1c.

15. The test support apparatus according to claim 1, wherein the abnormal tendency is determined based on a postprandial hyperglycemic spike.

16. The test support apparatus according to claim 15, wherein the processor is configured to
determine the test candidate based on meal content of the user.

17. The test support apparatus according to claim 1, wherein the first biological information is at least one of a heart rate, a blood pressure, respiration, an electrocardiogram, maximum oxygen intake, arterial oxygen saturation, or a body temperature, and
the second biological information is a diagnosis result of a disease of the user.

18. The test support apparatus according to claim 1, wherein the processor is configured to
perform a notification according to a monitoring result of the first biological information in a case where it is determined that there is no abnormal tendency in the first biological information.

19. The test support apparatus according to claim 1, wherein the first biological information is acquired by a measurement device that the user wears.

20. The test support apparatus according to claim 19, wherein the measurement device is a wearable measurement device.

21. The test support apparatus according to claim 19, wherein the processor is configured to
perform a notification for urging the user to wear the measurement device for a longer time in a case where whether or not there is an abnormal tendency in the first biological information is not determined.

22. The test support apparatus according to claim 1, further comprising a sensor that acquires the first biological information.

23. The test support apparatus according to claim 22, wherein the test support apparatus is a wearable apparatus.

24. The test support apparatus according to claim 1, wherein the action candidates further include a recommended action for the user.

25. The test support apparatus according to claim 1, wherein the processor is configured to
acquire the second biological information, and
determine whether to continue monitoring of the first biological information according to the acquired second biological information.

26. A test support method comprising:
determining whether or not there is an abnormal tendency in first biological information of a user by monitoring the first biological information;
determining, as a plurality of action candidates to be recommended for the user, a plurality of action candidates including at least one test candidate for acquiring second biological information associated with the first biological information in a case where there is the abnormal tendency in the first biological information; and
presenting the plurality of action candidates to the user.

27. A non-transitory computer-readable storage medium storing a test support program causing a computer to execute:
a procedure of determining whether or not there is an abnormal tendency in first biological information of a user by monitoring the first biological information;
a procedure of determining, as a plurality of action candidates to be recommended for the user, a plurality of action candidates including at least one test candidate for acquiring second biological information associated with the first biological information in a case where there is the abnormal tendency in the first biological information; and
a procedure of presenting the plurality of action candidates to the user.
